(19) United States
(12) Patent Application Publication
(10) Pub. No.: US 2017/0119660 A1
(43) Pub. Date: May 4, 2017

(54) PHARMACEUTICAL COMPOSITIONS FOR TRANSMUCOSAL DELIVERY

(71) Applicant: Solubest Ltd, Ness Ziona (IL)

(72) Inventors: Galia TEMTSIN-KRAYZ, Ness Ziona (IL); Sabina GLOZMAN, Ness Ziona (IL); Pavel KAZHDAN, Ness Ziona (IL)

(21) Appl. No.: 15/337,251

(22) Filed: Oct. 28, 2016

Related U.S. Application Data
(60) Provisional application No. 62/247,996, filed on Oct. 29, 2015.

(51) Int. Cl. A61K 9/00; A61K 9/16; A61K 9/20; A61K 31/352; A61K 31/573; A61K 38/28; A61K 31/4045; A61K 31/05

(52) U.S. Cl. CPC A61K 9/006; A61K 31/4045; A61K 9/1641; A61K 9/1652; A61K 9/1623; A61K 9/1611; A61K 31/05; A61K 9/1635; A61K 31/352; A61K 31/573; A61K 38/28; A61K 9/2031; A61K 9/2054; A61K 9/2059; A61K 9/2018; A61K 9/2009

(57) ABSTRACT

A pharmaceutical composition is provided for transmucosal administration of an active lipophilic compound through the oral mucosa comprising a lipophilic active compound, a polymeric matrix formed by two or more water-soluble polymers and a rapid dissolution agent. At least one of the water-soluble polymers is an amphiphilic polymer and at least one is either a hydrophilic polymer or an amphiphilic polymer with a hydrophobic-hydrophilic balance different from the first amphiphilic polymer. In addition, the polymeric matrix is not crosslinked and no covalent interaction occurs between the two or more polymers and between the polymers and the lipophilic active compound, which is interwoven with the aforesaid polymeric matrix.

### TECHNICAL FIELD

The present invention relates to oral delivery and, more particularly, to oral mucosal delivery of pharmaceutical compounds.

### BACKGROUND

Modern therapeutics includes a consideration of routes of administration and drug delivery in assessing therapeutic efficacy. Between the two major classifications of administration of drugs, local versus systemic, systemic administration approaches being less invasive are often preferred due to ease of administration. Systemic administration permits the administration of pharmaceutical compounds directly into the circulatory system so that the entire body is affected. This is in contrast with topical administration where the effect is generally local.

There are two routinely used systemic administration approaches: parenteral administration including intravenous and intraperitoneal injection, infusion or implantation, and enteral administration including oral drug delivery and drug delivery through the gastrointestinal tract. Transmucosal, particularly oral mucosal drug delivery, is an alternative method of systemic drug delivery that offers several advantages over both injectable and enteral methods.

The first-pass effect, which is also known as first-pass metabolism or pre-systemic metabolism, constitutes a serious problem encountered during the process of the oral drug delivery. It relates to a phenomenon of drug metabolism wherein the concentration of a drug is greatly reduced before it reaches the circulatory system. This phenomenon of losing a fraction of drug is observed due to absorption of the drug that occurs in the liver and gut walls. However, because the oral mucosa is highly vascularized, drugs that are absorbed through the oral mucosa directly enter the circulatory system bypassing the gastrointestinal tract and first-pass metabolism in the liver. Therefore, in order to avoid the first-pass effect and allow specific drugs to be absorbed directly into the circulatory system, alternative administration approaches, such as suppository, intravenous, intramuscular, aerosol inhalational, transdermal, transmucosal and sublingual routes can be used.

In view of the above, the transmucosal delivery of pharmaceutical compounds is a very attractive route of systemic administration. It avoids the first-pass effect and invasive injections to deliver the new and existing therapeutic drugs and pharmaceutical compounds systemically. In addition, oral transmucosal compositions are easy to administer and increase patient compliance. Several oral transmucosal products have been approved by the Food & Drug Administration (FDA), such as asenapine, buprenorphine, ergotamine, fentanyl, hydergine, isosorbide dinitrate, miconazole, nitroglycerin, ondansetron, testosterone, zolpidem, zuplenz and others.

The following factors are known to affect the transmucosal delivery: bioavailability; absorption rates; mucoadhesion, i.e., the adhesion between two materials at least one of which is a mucosal surface, leading to retention in the oral cavity; and pharmacokinetics. These factors may depend on a particular drug, the formulation and dosage used, and the particular site in the oral cavity where the drug is applied. Oral mucosa slightly varies between the sites of application, whether buccal, sublingual, or palatal. The transmucosal administration mode therefore depends upon the rate of vascularization, surface area, and other factors. Mucosal penetration is mediated by either an intercellular path, suitable mainly for hydrophilic drugs, or an intracellular path, suitable mainly for hydrophobic drugs.

Thus, oral mucosal or transmucosal delivery of drugs involves bypassing the gastrointestinal tract and first-pass metabolism in the liver by dissolution and absorption through the oral mucosa, typically under the tongue, sublingually or buccally through the inner mucosa of the cheeks. In contrast to other methodologies using sprays or patches, the transmucosal delivery, either sublingual or buccal, makes it possible rapidly disintegrating tablets or films, and is typically superior in obtaining patient compliance over other drug delivery systems.

In spite of many advantages, the transmucosal delivery of pharmaceutical compounds or drugs has some limitations. Rathbone et al (2015) in M. J. Rathbone et al (eds.), “Overview of Oral Mucosal Delivery”, Oral Mucosal Drug Delivery and Therapy, Advances in Delivery Science and Technology, 2015, pp 17-28, reviews the last-decades development in the field and outlines the following scientific findings:
(i) Oral cavity has smaller absorptive surface (˜214 cm²) area than the small intestines, and therefore, the amount of delivered drug is small (not more than 10 mg or 20 mg) per application;
(ii) Any drug should be completely soluble in saliva in order to be absorbed, but lipophilic enough in order to diffuse through lipophilic oral mucosa;
(iii) Bitter taste of the drug should be avoid or masked;
(iv) Saliva has slightly acidic pH, and therefore, the pH value of formulations must be in the range of 5-8;
(v) Any formulation must be safe, i.e. it should not cause the irritation or damage to sublingual, buccal and other oramucosal tissues;
(vi) Drugs have variable permeability, since the membrane thickness varies from a few hundred micrometres for the sublingual region to 500 μm for the buccal mucosa;
(vii) An excess of salivation and movements of the mouth or tongue may dislodge the dosage form from the site of administration and even cause the drug swallowing, and therefore, the mucoadhesive components are required;
(viii) Mucoadhesivity of the formulation can delay the drug release and absorption, and as a result, the fast onset will not be achieved.

The above limitations and challenges explain why the limited number of drugs has been formulated for the oramucosal delivery. Therefore, there is a long-felt need for a new transmucosal delivery system that would overcome the aforementioned limitations.

### SUMMARY

The present application describes embodiments of a pharmaceutical composition for transmucosal administration of an active lipophilic compound through the oral mucosa, said composition comprising:
(a) a lipophilic active compound;
(b) a polymeric matrix formed by two or more water-soluble polymers, wherein
   (i) at least one of said two or more water-soluble polymers is an amphiphilic polymer and at least one other of said two or more water-soluble polymers is either a hydrophilic polymer or an amphiphilic polymer with a hydrophobic-hydrophilic balance different from the first amphiphilic polymer; and
   (ii) said polymeric matrix is not crosslinked and no covalent interaction occurs between the two or more polymers and between the polymers and the lipophilic active compound, which is interwoven with said polymeric matrix; and
(c) a rapid dissolution agent.

The pharmaceutical composition of an embodiment of the present application contains the lipophilic active compound selected from analgesics, anti-inflammatory agents, antihelminthics, anti-arrhythmic agents, anti-bacterial agents, anti-viral agents, anti-coagulants, anti-depressants, anti-diabetics, anti-epileptics, anti-fungal agents, anti-gout agents, anti-hypertensive agents, anti-malarials, anti-migraine agents, anti-muscarinic agents, anti-neoplastic agents, chemotherapeitic drugs, antiproliferative, erectile dysfunction improvement agents, immunosuppressants, anti-protozoal agents, anti-thyroid agents, anxiolytic agents, sedatives, hypnotics, neuroleptics, .beta.-blockers, cardiac inotropic agents, corticosteroids, diuretics, anti-parkinsonian agents, gastro-intestinal agents, histamine receptor antagonists, keratolyptics, lipid regulating agents, anti-anginal agents, Cox-2 inhibitors, leukotriene inhibitors, macrolides, muscle relaxants, nutritional agents, opiod analgesics, protease inhibitors, sex hormones, stimulants, muscle relaxants, anti-osteoporosis agents, anti-obesity agents, cognition enhancers, anti-urinary incontinence agents, anti-benign prostate hypertrophy agents, essential fatty acids, non-essential fatty acids, and mixtures thereof.

In a particular embodiment, the lipophilic active compound is acetretin, acyclovir, albendazole, albuterol, almotriptan, aminoglutethimide, amiodarone, amlodipine, amphetamine, amphotericin B, amprenavir, aprepitant, atorvastatin, atovaquone, azithromycin, aztreonum, baclofen, beclomethasone, benezepril, benzonatate, betamethasone, bicalutanide, budesonide, bupropion, busulfan, butenafine, calcifediol, calcipotriene, calcitriol, camptothecin, candesartan, cannabidiol, capsaicin, carbamezepine, carotenes, cefixime, cefuraxime axetil, celecoxib, cerivastatin, cetirizine, chlorpheniramine, cholecalciferol, cilostazol, cimetidine, cinnarizine, ciprofloxacin, cisapride, clarithromycin, clemastine, clomiphene, clomipramine, clopidogrel, codeine, coenzyme Q10, cyclobenzaprine, cyclosporin, danazol, dantrolene, dexchlor-pheniramine, diclofenac, dicoumarol, digoxin, dehydroepiandrosterone, dihydro-ergotamine, dihydrotachysterol, dirithromycin, donezepil, enlimomab, efavirenz, eletriptan, eprosartan, ergocalciferol, ergotamine, essential fatty acid sources, etodolac, etoposide, famotidine, cannabidiol, fentanyl, fexofenadine, finasteride, fluconazole, flurbiprofen, fluvastatin, fosphenyloin, frovatriptan, fuirazolidone, gabapentin, gemfibrozil, glibenclamide, glipizide, glyburide, glimepiride, griseofulvin, halofantrine, hydrocortizone, ibuprofen, indinavir, irbesartan, irinotecan, isosorbide dinitrate, isotretinoin, itraconazole, ivermectin, ketoconazole, ketorolac, lamotrigine, lansoprazole, leflunomide, lidocaine, lisinopril, loperamide, loratadine, lovastatin, L-thryroxine, lutein, lycopene, medroxyprogesterone, mifepristone, mefloquine, megestrol, methadone, methoxsalen, metronidazole, miconazole, midazolam, miglitol, minoxidil, mitoxantrone, montelukast, morphine, nabumetone, nalbuphine, naratriptan, nelfinavir, nifedipine, nilsolidipine, nilutanide, nitrofurantoin, nizatidine, omeprazole, oprevelkin, oestradiol, oxaprozin, oxibutonine, paclitaxel, paracalcitol, paroxetine, pantoprazole, pentazocine, pioglitazone, pizofetin, phenoxymethyl penicillin, pravastatin, prednisolone, probucol, progesterone, propofol, pseudoephedrine, pyridostigmine, rabeprazole, raloxifene, rofecoxib, repaglinide, rifabutine, rifapentine, rimexolone, ritanovir, rizatriptan, rosiglitazone, saquinavir, sertraline, sibutramine, sildenafil, simvastatin, sirolimus, spironolactone, sumatriptan, svitriptan, tacrine, tacrolimus, tamoxifen, tamsulosin, targretin, tazarotene, telmisartan, teniposide, terbinafine, terazosin, tetrahydrocannabinol, tiagabine, ticlopidine, tirofibran, tizanidine, topiramate, topotecan, toremitfene, tramadol, tretinoin, troglitazone, trovafloxacin, ubidecarenone, valsartan, venlafaxine, verteporfin, vigabatrin, vitamin A, vitamin D, vitamin E, vitamin K, zafirlukast, zileuton, zolmitriptan, zolpidem or zopiclone, and pharmaceutically acceptable salts, isomers, and mixtures thereof.

In a specific embodiment, the lipophilic active compound is a cannabinoid selected from tetrahydrocannabinol (THC) and cannabidiol (CBD); or a triptan drug selected from almotriptan, eletriptan, frovatriptan, naratriptan, rizatriptan, sumatriptan, svitriptan, zolmitriptan, fentanyl, morphine, oxibutonine, tramadol, aprepitant, testosterone, sildenafil, prednisolone, insulin and glucagon.

In another particular embodiment, the pharmaceutical composition contains the rapid dissolution agent selected from mannitol, stevinol and a mixture thereof. In case the pharmaceutical composition of an embodiment contains a lipophilic active compound in a base form, it may further comprise a buffering agent, such as KH₂PO₄, which is added to the rapid dissolution agent in order to adjust the pH value of the composition to pH below 8, preferably to neutral physiological pH of 6.5-7.5. This is in contrast to the known state of the art where the salt form of the lipophilic active compound is used to improve solubility.

The pharmaceutical composition of an embodiment of the present application contains the amphiphilic polymer selected from the group consisting of polyethylene oxide (PEO), PEO derivatives, poloxamers, poloxamines, polyvinylpyrrolidones (PVP), hydroxypropyl cellulose, hypromellose, hypromellose phthalate, hypromellose acetate succinate, polyacrylates, polymethacrylates, polyethylene glycol (PEG) copolymers, PEO/polypropylene glycol copolymers, PEG-modified starches, vinyl acetate-vinyl pyrrolidone copolymers, polyacrylic acid copolymers, polymethacrylic acid copolymers, plant proteins and protein hydrolysates.

In a further embodiment, the transmucosal pharmaceutical composition contains the hydrophilic polymer selected from the group consisting of starch, soluble starch, sodium carboxymethylcellulose (NaCMC), hydroxyethylcellulose, polyvinyl alcohol, sodium alginate, chitosan, and carrageenan.

In another aspect, the present application provides a method for the preparation of a composition of an embodiment, comprising the following steps:
i) preparing a clear and homogeneous solution of the two or more polymers, the rapid dissolution agent and the lipophilic active compound in water or in a mixture of water and one or more organic solvents; and
ii) drying the clear and homogeneous solution, preferably by spray drying, to form a dry powder.

In a particular embodiment, the clear and homogeneous solution of the two or more polymers in the first step above is obtained by adding the lipophilic active compound either as a solid base or as a salt dissolved in one or more organic solvents to an aqueous solution of the polymers and the rapid dissolution agent.

The pharmaceutical composition of an embodiment may further comprise one or more pharmaceutically acceptable carriers, excipients or both. In yet further embodiment, the pharmaceutical composition may be prepared in a form of a powder, simple powder mixtures, powder microspheres, coated powder microspheres, liposomal dispersions and combinations thereof. It may be formulated into a dosage form for oral administration selected from capsules, tablets, beads, grains, pills, granulates, granules, powder, pellets, sachets, troches, disks, films, oral suspensions and aerosol.

The pharmaceutical composition of an embodiment may be administered in a sublingual or buccal transmucosal solid dosage forms.

The details of one or more embodiments are set forth in the accompanying figures and the description below. Other features, objects and advantages of the described techniques will be apparent from the description and drawings and from the claims.

### BRIEF DESCRIPTION OF THE DRAWINGS

Disclosed embodiments will be understood and appreciated more fully from the following detailed description taken in conjunction with the appended figures.

FIG. 1 shows the dissolution profile of sumatriptan API (active pharmaceutical ingredient) from the Transmucosal™ formulation of an embodiment (see Example 3) (trinagles) vs unformulated sumatriptan (“Raw API”) in saliva (squares).

FIG. 2 shows the Franz diffusion cell experiment for testing in-vitro permeability through human buccal tissues of three sumatriptan samples suspended in 0.5 ml of artificial saliva at concentration 7.5 mg/ml:
(1) Unformulated sumatriptan (squares),
(2) Imitrex® nasal spray containing sumatriptan API (triangles), and
(3) TransmucosalTransmucosal formulation of an embodiment (see Example 3) (circles).

FIG. 3 shows the sumatriptan base flat-square sublingual tablets with active dose of 75 mg.

FIG. 4 shows the dissolution profile of sumatriptan base flat-square sublingual tablets with active dose of 25 mg (rhombs) and 75 mg (squares) in artificial saliva.

FIG. 5 shows the comparative dissolution profile of cannabidiol API from the CBD formulation of an embodiment with dose of 20 mg in 200 ml of Fasted State Simulating State Intestinal Fluid (FaSSIF) (see Example 13) (trinagles) vs unformulated cannabidiol in saliva (rhombs).

FIG. 6 shows the XRD (X-Ray Diffraction) spectra of aprepitant API from the formulation of an embodiment (Sapt-121-16) (see Example 16, upper spectrum) and unformulated aprepitant (lower spectrum).

FIG. 7 shows the dissolution profile of aprepitant API from the formulation of an embodiment (Sapt-121-16) (see Example 16) (squares) vs aprepitant API from the Emend® commercial grannular formulation, in which aprepitant is presented in a nanocrystalline form (rhombs), in a standart FDA approved media of 2.2% sodium lauryl sulfate.

FIG. 8 shows the dissolution profile of aprepitant API from the formulation of an embodiment (Sapt-121-16) in Fasted State Simulating State Intestinal Fluid (FaSSIF) (see Example 16) (squares) vs aprepitant API from the Emend® commercial grannular formulation, in which aprepitant is presented in a nanocrystalline form (rhombs).

FIG. 9 shows the pharmacokinetic profile of sumatriptan API (the means of plasma sumatriptan values of three volunteers) following administration of sumatriptan sublingual tablet vs Imitrex® in a crossover clinical trial.

FIG. 10 shows the pharmacokinetic profile of sumatriptan API (the separated profiles for each volunteer) following administration of sumatriptan sublingual tablet vs Imitrex® in a crossover clinical trial.

### DETAILED DESCRIPTION

In the following description, various aspects of the present application will be described. For purposes of explanation, specific configurations and details are set forth in order to provide a thorough understanding of the present application. However, it will also be apparent to one skilled in the art that the present application may be practiced without the specific details presented herein. Furthermore, well-known features may be omitted or simplified in order not to obscure the present application.

The term “comprising”, used in the claims, should not be interpreted as being restricted to the components and steps listed thereafter; it does not exclude other components or steps. It needs to be interpreted as specifying the presence of the stated features, integers, steps or components as referred to, but does not preclude the presence or addition of one or more other features, integers, steps or components, or groups thereof. Thus, the scope of the expression “a composition comprising x and z” should not be limited to compositions consisting only of components x and z.

The present invention relates to transmucosal pharmaceutical compositions, in particular, oral mucosal pharmaceutical compositions, obtained by using the technology developed by the applicant and described in WO 2009/040818 and U.S. Pat. No. 9,254,268 ('268), in combination with the use of rapid dissolution agents and optionally pH-adjusting and taste masking agents. However, the composition of an embodiment of the present application imparts a drug an ability to be delivered into the blood through mucosal cavity, in contrast to the composition of '268. The composition of '268 has proven to reach much better bio-absorption in gastrointestinal cavity, without any possibility to be used directly as a trans-mucosal delivery system.

In one embodiment, the invention relates to a pharmaceutical composition for transmucosal administration of an active lipophilic compound through the oral mucosa, said composition comprising:
(a) a lipophilic active compound;
(b) a polymeric matrix formed by two or more water-soluble polymers,
wherein
   (i) at least one of said two or more water-soluble polymers is an amphiphilic polymer and at least one other of said two or more water-soluble polymers is either a hydrophilic polymer or an amphiphilic polymer with a hydrophobic-hydrophilic balance different from the first amphiphilic polymer; and
   (ii) said polymeric matrix is not crosslinked and no covalent interaction occurs between the two or more polymers and between the polymers and the lipophilic active compound, which is interwoven with said polymeric matrix; and
(c) a rapid dissolution agent.

In another embodiment, the lipophilic active compound may be delivered in a non-ionised form. If the lipophilic active compound has a basic or acidic nature, then the composition of an embodiment should contain the pH-adjusting and buffering agent.

The lipophilic active compound may be selected from analgesics, anti-inflammatory agents, antihelminthics, anti-arrhythmic agents, anti-bacterial agents, anti-viral agents, anti-coagulants, anti-depressants, anti-diabetics, anti-epileptics, anti-fungal agents, anti-gout agents, anti-hypertensive agents, anti-malarials, anti-migraine agents, anti-muscarinic agents, anti-neoplastic agents, chemotherapeitic drugs, antiproliferative, erectile dysfunction improvement agents, immunosuppressants, anti-protozoal agents, anti-thyroid agents, anxiolytic agents, sedatives, hypnotics, neuroleptics, .beta.-blockers, cardiac inotropic agents, corticosteroids, diuretics, anti-parkinsonian agents, gastro-intestinal agents, histamine receptor antagonists, keratolyptics, lipid regulating agents, anti-anginal agents, Cox-2 inhibitors, leukotriene inhibitors, macrolides, muscle relaxants, nutritional agents, opiod analgesics, protease inhibitors, sex hormones, stimulants, muscle relaxants, anti-osteoporosis agents, anti-obesity agents, cognition enhancers, anti-urinary incontinence agents, anti-benign prostate hypertrophy agents, essential fatty acids, non-essential fatty acids, and mixtures thereof.

In certain embodiments, the lipophilic active compound may be acetretin, acyclovir, albendazole, albuterol, almotriptan, aminoglutethimide, amiodarone, amlodipine, amphetamine, amphotericin B, amprenavir, aprepitant, atorvastatin, atovaquone, azithromycin, aztreonum, baclofen, beclomethasone, benezepril, benzonatate, betamethasone, bicalutanide, budesonide, bupropion, busulfan, butenafine, calcifediol, calcipotriene, calcitriol, camptothecin, candesartan, cannabidiol, capsaicin, carbamezepine, carotenes, cefixime, cefuraxime axetil, celecoxib, cerivastatin, cetirizine, chlorpheniramine, cholecalciferol, cilostazol, cimetidine, cinnarizine, ciprofloxacin, cisapride, clarithromycin, clemastine, clomiphene, clomipramine, clopidogrel, codeine, coenzyme Q10, cyclobenzaprine, cyclosporin, danazol, dantrolene, dexchlor-pheniramine, diclofenac, dicoumarol, digoxin, dehydroepiandrosterone, dihydro-ergotamine, dihydrotachysterol, dirithromycin, donezepil, enlimomab, efavirenz, eletriptan, eprosartan, ergocalciferol, ergotamine, essential fatty acid sources, etodolac, etoposide, famotidine, cannabidiol, fentanyl, fexofenadine, finasteride, fluconazole, flurbiprofen, fluvastatin, fosphenyloin, frovatriptan, fuirazolidone, gabapentin, gemfibrozil, glibenclamide, glipizide, glyburide, glimepiride, griseofulvin, halofantrine, hydrocortizone, ibuprofen, indinavir, irbesartan, irinotecan, isosorbide dinitrate, isotretinoin, itraconazole, ivermectin, ketoconazole, ketorolac, lamotrigine, lansoprazole, leflunomide, lidocaine, lisinopril, loperamide, loratadine, lovastatin, L-thryroxine, lutein, lycopene, medroxyprogesterone, mifepristone, mefloquine, megestrol, methadone, methoxsalen, metronidazole, miconazole, midazolam, miglitol, minoxidil, mitoxantrone, montelukast, morphine, nabumetone, nalbuphine, naratriptan, nelfinavir, nifedipine, nilsolidipine, nilutanide, nitrofurantoin, nizatidine, omeprazole, oprevelkin, oestradiol, oxaprozin, oxibutonine, paclitaxel, paracalcitol, paroxetine, pantoprazole, pentazocine, pioglitazone, pizofetin, phenoxymethyl penicillin, pravastatin, prednisolone, probucol, progesterone, propofol, pseudoephedrine, pyridostigmine, rabeprazole, raloxifene, rofecoxib, repaglinide, rifabutine, rifapentine, rimexolone, ritanovir, rizatriptan, rosiglitazone, saquinavir, sertraline, sibutramine, sildenafil, simvastatin, sirolimus, spironolactone, sumatriptan, svitriptan, tacrine, tacrolimus, tamoxifen, tamsulosin, targretin, tazarotene, telmisartan, teniposide, terbinafine, terazosin, tetrahydrocannabinol, tiagabine, ticlopidine, tirofibran, tizanidine, topiramate, topotecan, toremitfene, tramadol, tretinoin, troglitazone, trovafloxacin, ubidecarenone, valsartan, venlafaxine, verteporfin, vigabatrin, vitamin A, vitamin D, vitamin E, vitamin K, zafirlukast, zileuton, zolmitriptan, zolpidem or zopiclone, and pharmaceutically acceptable salts, isomers, and mixtures thereof.

In a particular embodiment, the lipophilic active compound is a cannabinoid selected from tetrahydrocannabinol (THC) and cannabidiol (CBD); a triptan drug selected from almotriptan, eletriptan, frovatriptan, naratriptan, rizatriptan, sumatriptan, svitriptan, and zolmitriptan; fentanyl salts; lidocaine salts; morphine sulfate; oxibutonine salts; pentazocine salts; sildenafil salts; and tramadol salts.

Any rapid dissolution agent known in the art can be used according to the embodiments of the present application. In certain embodiments, the rapid dissolution agent is mannitol, stevinol, PVP, EDTA or a mixture thereof. The rapid dissolution may be carried out with binders, pH adjusting buffers and taste-masking additives.

In case the pharmaceutical composition of an embodiment contains a lipophilic active compound in a base form, it may further comprise a buffering agent, such as KH₂PO₄, which is added to the rapid dissolution agent in order to adjust the pH value of the composition to pH below 8, preferably to neutral physiological pH of 6.5-7.5, thereby allowing administration of the drug through the oral mucosa. This is in contrast to the known state of the art where the salt form of the lipophilic active compound is used to improve solubility. This is for example necessary when the active lipophilic compound is used as a free base or free acid. The use of the non-ionised active lipophilic compound or salt forms obviates the need for addition of the buffering agent.

In a further embodiment, the amphiphilic polymer may be polyethylene oxide (PEO), PEO derivatives, poloxamers (preferably, Poloxamer 407), poloxamines, polyvinylpyrrolidones (PVP), hydroxypropyl cellulose, hypromellose, hypromellose phthalate, hypromellose acetate succinate, polyacrylates, polymethacrylates, polyethylene glycol (PEG) copolymers, PEO/polypropylene glycol copolymers, PEG-modified starches, vinyl acetate-vinyl pyrrolidone copolymers, polyacrylic acid copolymers, polymethacrylic acid copolymers, plant proteins and protein hydrolysates.

In yet further embodiment, the hydrophilic polymer may be starch, soluble starch, sodium carboxymethylcellulose (NaCMC), hydroxyethylcellulose, polyvinyl alcohol, sodium alginate, chitosan, and carrageenan.

In an exemplary embodiment, the following combinations of polymers may be used:
1) Two polymers form the polymeric matrix, one of the polymers is an amphiphilic polymer, preferably Poloxamer 407, and the other polymer is a hydrophilic polymer, preferably NaCMC;
2) Three polymers form the polymeric matrix, two of the polymers are amphiphilic polymers, preferably Poloxamer 407 and PVP, and the other polymer is a hydrophilic polymer, preferably NaCMC; or
3) Three polymers form the polymeric matrix, one of the polymers is an amphiphilic polymer, preferably Poloxamer 407, and the other two polymers are hydrophilic polymers, preferably NaCMC and soluble starch.

In another exemplary embodiment, the pharmaceutical composition of the present application is selected from the following:
1) Sumatriptan, Poloxamer 407, NaCMC and mannitol;
2) Sumatriptan, Poloxamer 407, NaCMC, mannitol and KH₂PO₄;
3) Sumatriptan, Poloxamer 407, NaCMC, soluble starch and mannitol;
4) Sumatriptan, Poloxamer 407, NaCMC, soluble starch, mannitol, stevinol and KH₂PO₄;
5) Cannabidiol, Poloxamer 407, NaCMC and mannitol;
6) Aprepitant, Poloxamer 407, NaCMC and mannitol;
7) Tetrahydrocannabinol, D-α-tocopherol, polyethylene glycol, Poloxamer 407, NaCMC, soluble starch and stevinol; and
8) Prednisolone, Poloxamer 407, NaCMC and mannitol; and
9) Insulin, EDTA, Poloxamer 407 and NaCMC.

In another aspect, the present application provides a method for the preparation of a composition of an embodiment, comprising the following steps:
i) preparing a clear and homogeneous solution of the two or more polymers, the rapid dissolution agent and the lipophilic active compound in water or in a mixture of water and one or more organic solvents; and
ii) drying the clear and homogeneous solution, preferably by spray drying, to form a dry powder.

The polymers-lipophilic drug clear and homogeneous solution can be prepared in various ways according to the polymers used. The lipophilic drug can be dissolved in at least one organic solvent that is miscible with water and does not lead to precipitation of the polymers when the organic solution containing the lipophilic drug is added to the polymers solution. Examples of such solvents include, but are not limited to, acetic acid, acetonitrile, acetone, 1-butanol, 2-butanol, N,N-dimethylacetamide; N,N-dimethylformamide, dimethyl sulfoxide, 1,4-dioxane, ethanol, formic acid, methanol, 3-methyl-1-butanol, methylethyl ketone, 2-methyl-1-propanol, 1-methyl-2-pyrrolidone, 1-pentanol, n-propanol, 2-propanol and tetrahydrofuran. In certain embodiments, the organic solvent is n-propanol, ethanol, 1-vinyl-2-pyrrolidone or acetonitrile, or a mixture of n-propanol and acetone, or ethanol and water.

In another embodiment, the lipophilic active compound is added in a solid form, not in organic solution, to the aqueous solution of the polymers (solvent-free preparation). This is usually possible when the lipophilic active compound has sufficient solubility in the buffer-polymer solution. For example, the lipophilic active compound present in the form of a base has sufficient solubility in the buffer-polymer solution. Otherwise, it requires dissolution in organic solvents. Thus, the clear and homogeneous solution of step (i) can be obtained by adding the lipophilic active compound either in a solid form, or dissolved in one or more organic solvents, to an aqueous solution of the polymers and rapid dissolution agent. Any conventional method known for drying solutions such as spray drying, evaporation by heating under vacuum, and freeze-drying, can be used according to embodiments of the present application. In preferred specific embodiment, the powder composition is prepared by the spray drying method.

The pharmaceutical composition of an embodiment may further comprise one or more pharmaceutically acceptable carriers, excipients or both. In certain embodiments, the pharmaceutical composition may further comprise a disintegration agent, such as cross-linked starch, crosscarmellose sodium or crosspovidone, which is added for example, to a tablet to induce breakup, when the tablet comes in contact with aqueous medium. In other embodiments, pharmaceutical composition may further comprise a taste masking agent selected from sweeteners, essential oils and common flavors, for example, the combination of sucralose, stivenol, menthol and optionally vanillin, added in order to mask the bitter test of the lipophilic active compound. The pharmaceutical composition of an embodiment may further comprise the tableting binders and lubricants, such as microcrystalline cellulose and magnesium stearate, if the disintegration agent is added.

In yet further embodiment, the pharmaceutical composition may be prepared in a form of a powder, simple powder mixtures, powder microspheres, coated powder microspheres, liposomal dispersions and combinations thereof. It may be formulated into a dosage form for oral administration selected from capsules, tablets, beads, grains, pills, granulates, granules, powder, pellets, sachets, troches, disks, films, oral suspensions and aerosol. The pharmaceutical composition of an embodiment may be administered in a sublingual or buccal transmucosal solid dosage forms.

Combination of the unique amphiphilic-hydrophilic polymers-drug matrix described in the present application provides a sufficient delay in the absorption of the drug inside the mouth. As a result, the lipophilic drug reaches the maximum concentration in the saliva very fast that significantly increases its permeability rate providing maximum therapeutic efficacy. This is very surprising finding, particularly since the compositions of an embodiment contain carboxymethyl cellulose sodium and starch, which are well known for their mucoadhesive and swelling properties and generally provide an extended and slow release of the drugs.

The oral transmucosal compositions of the invention can be used in many clinical indications, for example, in the treatment of migraines. Nonsteroidal anti-inflammatory drugs (NSAIDs) and triptans are used as a first line of treatment for migraine attacks to reduce pain and restore function. Triptan-type migraine medications, acting through a serotonin receptor, constrict blood vessels during a migraine attack and relieve migraine-related symptoms such as pain. However, the delivery route is very important for the onset of action by triptans. For example, intranasal sprays usually act within 10 to 15 minutes and are therefore, the most rapid and effective treatment, but many patients do not like their taste or can have sinusitis that changes the drug effectiveness. Orally disintegrating triptans have a similar onset of action and efficacy to oral tablets and a particular advantage in patients with nausea-associated migraine attacks. Ergotamine, a serotonin receptor specific vasoconstrictor, is another anti-migraine drug taken in small dosages, but unfortunately, it has a very low oral bioavailability.

At least 5% systemic absorption is needed to provide any benefit. The present invention allows such an absorption rate. Many other out-of-label prescription drugs, including beta-blockers such as propranolol; anticonvulsants, such as gabapentine, carbamazepine valproate, carbamazepine; anti-depressants such as amitryptilines and others; anti-inflammatory compounds, such as steroids, NSAIDS, lidocaine and derivatives thereof; and calcium channel blockers, such as verapamilar, are used to prevent migraine attacks, and are suitable candidates as the active compounds for the pharmaceutical compositions of the present application.

The delivery system of the invention may also be used for the transmucosal delivery of pharmaceutical compounds such as oxybutynin, tolterodine, trospium, solifenacin, and darifenacin, to other parts of the body, for example, for the treatment of an overactive bladder. Using transmucosal oxybutynin results in an escape from the first-pass hepatic metabolism and conversion of the oxybutynin to N-desethyloxybutynin.

Several advantages of the transmucosal composition of the present application include avoiding the hostile gastrointestinal (GI) environment, bypassing the first-pass effect (liver metabolism), having a high rate of vascularization coupled to relatively high penetrability, having high cellular turnover rates, and making it possible to use potent, low-dosage drugs. This may be useful for enzyme-liable drugs, such as peptides, for example insulin or growth hormone.

Regrading the first-pass effect, steroids, such as prednisone, prednisolone, cortisone, cortisol and triamcinolone, androgenic steroids, such as methyltesterone, testosterone, and fluoxmesterone, estrogenic steroids and progestational steroids, such as progesteroneare, are an example of drugs that undergo a significant amount of the first-pass metabolism when administered orally. As mentioned above, the pharmaceutical compositions of an embodiment containing, for example, the aforementioned steroids and used for transmucosal administration of said steroids through the oral mucosa are capable of bypassing the first-pass metabolism.

The pharmaceutical compositions of an embodiment may also be useful in certain applications where the active compound is to be delivered to systemic exposure through mucosa, e.g. vaccination. The exemplary immunological agents in that case include immune globulins, monoclonal antibody agents, antivenins, agents for active immunization, allergenic extracts, immunologic agents, and anti-rheumatic agents.

The pharmaceutical compositions of an embodiment can deliver the lipophilic active compounds, such as antiemetic drugs aprepitant and granisteron, as well as various chemotherapy agents, to the patient's circulatory system, even if the patient has certain swallow difficulties due to his/her age, esophagitis, CNS disorders, chemotherapy induced nausea vomiting, etc. Many people with neurodegenerative disorders (for example, patients taking anti-Parkinson agents, such as biperiden, carbidopa, levodopa, ropinirole, rasagiline, pramipexole, entacapone, benzacide, bromocriptine, selegiline, amantadine, tolcapone, trihexylphenidyl and medical cannabinoids, or anti-dementia and anti-Alzheimer drugs, such are memantine, donepexil, galantamine, rivastigmine, and tacrine) will also benefit from significantly more compliant formulations of the present application. In addition, it should be noted that the transmucosal proton pump inhibitors (PPI) administered with the formulations of the present application may effectively control intragastric pH. This can be an alternative to intravenous or intranasal tubes administered PPIs to those patients who cannot swallow solid-dose formulations.

The pharmaceutical composition of an embodiment can be administered to a patient when the lipophilic active compound must be delivered very fast. The non-limiting examples of fast onset, or on-demand-needed medications are anti-pain, anti-psychotic, anxiolytic, anti-seizure, cardio-protective, anti-stroke, anti-emetic, anti-narcoleptic and anti-dot drugs. The examples of the pharmaceutical compounds with a therapeutic efficacy due to fast onset of action or and on-demand use are antipsychotics, such as fluphenazine, prochlorperazine, perphenazine, lithium carbonate, lithium citrate, thioridazine, molindone, trifluoperazine, amitriptyline, trifluopromazine, chlorpromazine, clozapine, haloperidol, loxapine, mesoridazine, olanzapine, quetiapine, ziprasidone, risperidone, chlorprothixene, pimozide, mesoridazine besylate and thiothixene.

Analgesic drugs used in the pharmaceutical composition of an embodiment are for example, etorphine, diflunisal, aspirin, ibuprofen, profen-type compounds, morphine, codeine, levorphanol, hydromorphone, oxymorphone, oxycodone, hydrocodone, naloxene, nalorphine, levallorphan, fentanyl, bremazocine, meperidine, tramadol and acetaminophen. Antihistamines formulated in the pharmaceutical composition of an embodiment are, for example, acrivastine, astemizole, ebastine, norastemizol, brompheniramine, cetirizine, clemastine, fexofenadine, diphenhydramine, famotidine, meclizine, nizatidine, perilamine and promethazine. Anti-asthma drugs included in the pharmaceutical compositions of an embodiment are theophylline, ephedrine, dipropionate, epinephrine and beclomethasone. Anticoagulants are heparin, bishydroxycoumarin and warfarin. Psychic energizers used in the pharmaceutical composition of an embodiment are parglyene, isocoboxazid, nialamide, phenelzine, imipramine and tranylcypromine Anticonvulsants are primidone, clonazepam, phenobarbital, mephobarbital, diphenylhydantion, enitabas, ethltion, pheneturide, valproic acid, ethosuximide, diazepam, phenytoin, carbamazepine, topiramate, felbamate, tiagabine levetiracetam, lamotrigine, lorazepam, oxcarbazepine, chlorazepate, gabapentin and zonisamide. Anti-spasmodic drugs formulated in the pharmaceutical composition of an embodiment are muscle contractants, such as atropine, scopolamine, methscopolamine, oxyphenonium, papaverine, and prostaglandins.

Muscle relaxants used in the pharmaceutical composition of an embodiment are alcuronium, alosetron, aminophylline, baclofen, carisoprodol, chlorphenesin, pridinol (pridinolum), chlorphenesin carbamate, chlorzoxazone, chlormezanone, dantrolene, decamethonium, dyphylline, eperisione, ethaverine, gallamine triethiodide, metaxalone, hexafluorenium, metocurine iodide, orphenadrine, pancuronium, papaverine, tizanidine, pipecuronium, biperiden theophylline, tolperisone, tubocurarine, succinylcholine-chloride, vecuronium, idrocilamide, ligustilide, cnidilide, senkyunolide, danbrolene, diazepam, cyclobenzaprine, methocarbamol, mephenesin, methocarbomal and trihexylphenidyl.

Sympathomimetic drugs introduced into the pharmaceutical compositions of an embodiment are albuterol, epinephrine, amphetamine ephedrine and norepinephrine. Cardiovascular drugs formulated into the pharmaceutical compositions of an embodiment are procainamide, nitroglycerin, beta.-blockers, such as caravedilol, pindolol, propranolol, practolol, metoprolol, esmolol, oxprenolol, timolol, atenolol, alprenolol, acebutolol and alpha-adrenergic receptors, such as terazosin, doxazosin, clonidine hydrochloride, prazosin and tamsulosin. Other life-save drugs, which can be formulated into the pharmaceutical compositions of an embodiment, are presented in the WHO Model List of Essential Medicines, for example glucagon hormone.

The transmucosal compositions of the invention may be used to address significant unmet medical needs, such as treating diabetes, chemotherapy-induced nausea, breakthrough pain, and acute psychotic and neurological disorders among others. For the treatment of diabetes, the composition may contain the transmucosal insulin to overcome the major disadvantage of oral dosage forms of insulin, i.e. the inherent variability of the GI tract absorption, in order to yield a supplementary dosage, which would be alternative to insulin injections. Thus, a patient may effectively control his or her glucose level via oral delivery of the transmucosal insulin practicing the present invention.

The pharmaceutical composition of an embodiment for transmucosal administration of an active lipophilic compound through the oral mucosa has the following properties:
1) Controlled adhesiveness to mucosal surface;
2) Improved solubility and high dissolution rate of a pharmaceutical compound in saliva;
3) Release of drug with a sufficient residual time for mucosal absorption;
4) Delivery of the drug in a non-ionizing lipophilic form in order to permeate series of barriers to reach the circulatory system via the most efficient trans-cellular route;
5) Improvement of the permeability properties, that together with the high dissolution rate and sufficient residual time, translates into earlier and higher drug exposure and faster absorption with a lower delivered dose than the oral tablet; therefore, the fast onset of action is observed;
6) Similarity of the earlier exposure to that of the nasal spray, yet the sublingual transmucosal form presents a higher initial peak and prolonged exposure compared with the intranasal formulation.
7) Ability to deliver the relatively high dose of drug, in order to provide both important features: fast onset of action and therapeutic effect prolongation. The former is a result of transmucosal absorption, and the latter is a result of the gastrointestinal absorption of the swallowed drug;
8) Improved bioavailability of the drug (75-mg sublingual tablet of invention showed the pharmacokinetics similar to 100-mg commercial oral tablet).

Both lipophilic and hydrophilic drugs can be effectively used in transmucosal delivery. Hydrophilic compounds penetrate the epithelial barrier of the oral mucosa by inter-cellular route, while lipophilic compounds are uptaken by the trans-cellular mechanism. The following equations (1) and (2) show the relationship between the drug flux through oral mucosa and other factors:

### Intracellular Delivery (1):

$$J = \frac{DEC}{h}$$

### Transcellular Delivery (2):

$$J = \frac{( 1 - E ) DCK}{h}$$

wherein:
J—drug flux
D—diffusion coefficient of the drug
E—Area fraction of the intercellular/transcellular route
C—donor drug concentration
K—drug partition coefficient
h—path length

The technology developed by the applicant in WO 2009/040818 made it possible to increase both the dissolution rate of a drug and its water solubility in various products. The following Noyes-Whitney equation (3) defines the dissolution rate of a drug:

### Noyes-Whitney Equation (3):

$$\frac{d W}{d t} = \frac{DA ( Cs - C )}{L}$$

wherein
dW/dt—dissolution rate of a solid compound (drug)
A—surface area of the solid compound
C—concentration of the drug in a solvent
Cs—concentration of the drug in the diffusion layer surrounding the solid compound
D—diffusion coefficient
L—diffusion layer thickness

Several parameters appearing in the Noyes-Whitney equation (3) can be maximized via formulation development. For example, the diffusion coefficient D can be increased by introducing the penetration enhancers into formulation. Rapid disintegration of the formulation and high dissolution rate of the drug are the key factors in the oral mucosal delivery. Achieving the high concentration of the drug in saliva C, and thereby, accelerating the drug flux, is possible through the reduction of the drug particle size. In fact, dissolution rate can be tremendously increased by increasing surface area A via particle size reduction. Increasing the drug dissolution rate will be beneficial both for the lipophilic and hydrophilic drug uptake; however, the increase of the drug solubility in saliva is a critical factor for the lipophilic API permeation.

It is known that the solubility (in any solvent) of a crystalline solute is at least partially dependent on certain properties of the crystal. The reduction in solubility that is attributable to solute crystallinity is given by the Hildebrand equation (4): Hildebrand equation (4):

$$log M = \frac{- Δ {S}_{f} ( {T}_{m} - T )}{2.303 RT}$$

wherein
M—Mole fraction solubility of a solid compound (drug), defined as follows:

$$M = \frac{moles of the solid compound}{moles of the solid compound + moles of solvent}$$

Tₘ and T—melting point of the solid compound and temperature of interest, respectively (both in ° K)
ΔSf—entropy of fusion of the solid compound
   Thus, solubility of the drug can be exponentially increased with decrease of melting point of the solid compound.

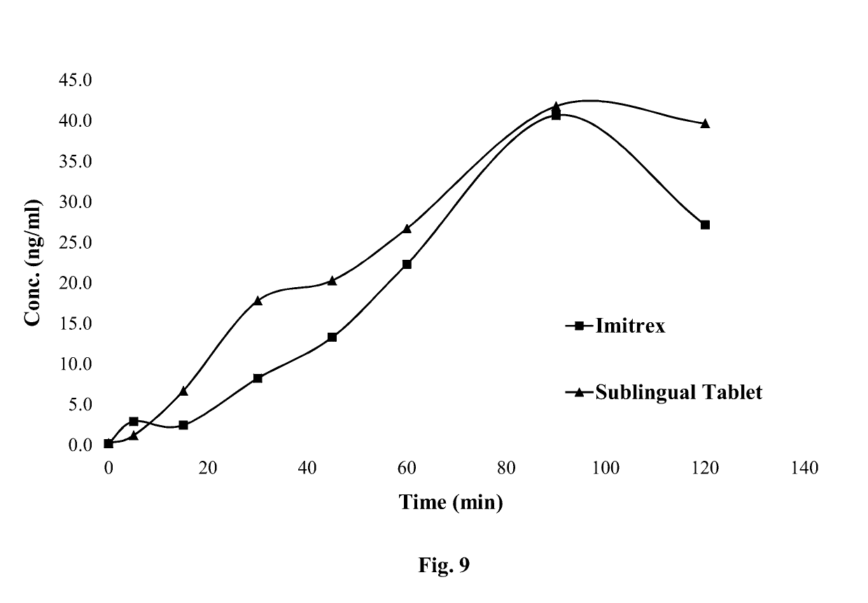

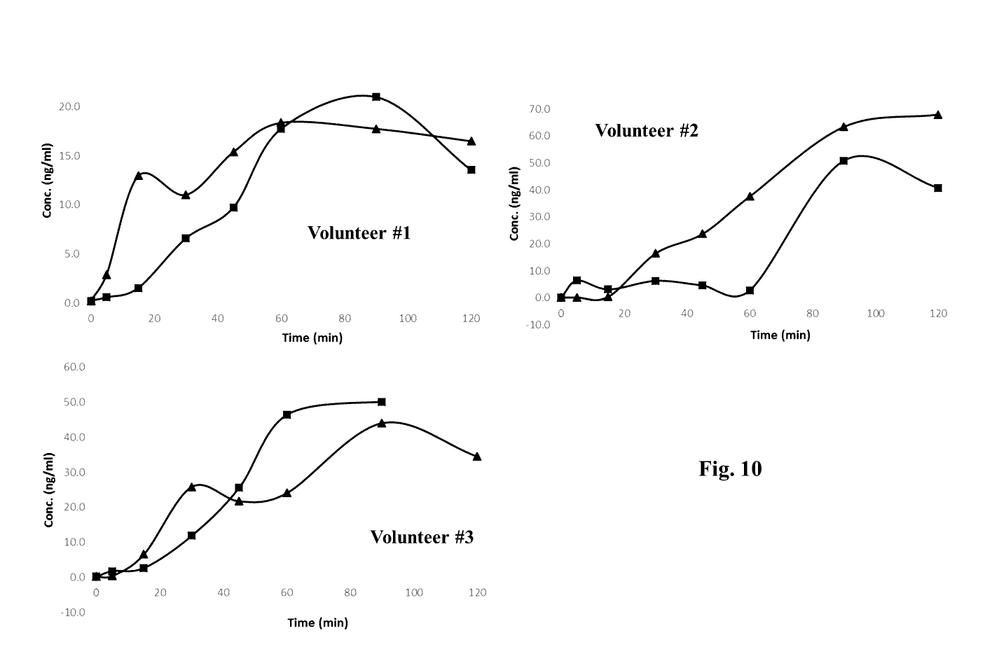

The obtained properties of the pharmaceutical composition of an embodiment were unexpected. The solid dispersion obtained by the applicants and described in WO 2009/040818 has only slightly delayed dissolution of the prepared dosage forms. Powders and granules exemplified in WO 2009/040818 are dissolved over 15 min, and tablets are dissolved over 60 min. To the contrary, the pharmaceutical composition of an embodiment of the present application formulated in granules reaches the complete dissolution within 2-3 min, while the formulation in tablets is completely dissolved within 30 min. The sublingual tablet however disintegrates within 5-7 min upon application. This relatively high dissolution rate is translated into shorter Tₘₐₓ values about 30 min (see Example 20 and FIGS. 9 and 10).

Other obtained important property of the pharmaceutical composition of an embodiment, which was unexpected, is improvement of the drug permeability. Previously, the applicants succeeded to achieve only solubility enhancement (see WO 2009/040818 for details). Incorporation of a third hydrophilic polymer into the lipophilic active compound-polymer matrix, according to embodiments of the present application, together with the rapid dissolution agent and buffering agent, surprisingly not only increased the dissolution rate of the active compound, but also enhanced the permeability through the oral mucosal membrane (see Examples 6 and 8 in the Examples section below).

The superiority of the pharmaceutical compositions of embodiments of the present application vis-a-vis the compositions of the previously developed technology described by the applicants in WO 2009/040818, can be demonstrated by ex-vivo Franz cell experiments and in-vivo pharmacokinetic studies (see the Examples section). The pharmaceutical composition of an embodiment comprises the powder consisting of the lipophilic drug-polymers complex and may further comprise one or more pharmaceutically acceptable inert carriers or excipients or both, such as taste-masking agents, penetration enhancers, binders, diluents, disintegrants, fillers, glidants, lubricants, suspending agents, sweeteners, essential oils, flavouring agents, buffers, wicking agents, wetting agents, and effervescent agents. The compositions of the invention show rapid dissolution in tests performed in accordance with the FDA Dissolution Methods for Drug Products. For lipophilic drugs having good permeability, wherein solubility is the main deterrent to achieve good bioavailability, the dissolution tests are indicative of solubility and therein bioavailability.

Administration of the pharmaceutical composition of an embodiment results in rapid dissolution, immediate release and improved bioavailability of the lipophilic drug. The term “bioavailability”, as used herein, refers to the degree to which the lipophilic drug becomes available to the target tissue after administration. A suitable bioavailability for the lipophilic drug composition of an embodiment ideally shows that the administration of such pharmaceutical composition results in a bioavailability that is improved (or is at least the same) compared to the bioavailability obtained after administration of the unformulated lipophilic drug or of a commercially available product containing the lipophilic drug in the same amounts. The term “unformulated” lipophilic drug, as used herein, refers to the lipophilic compound used as a raw crystalline powder.

The term “permeability”, as used herein, refers to permeability of drugs via oral mucosa (buccal and sublingual), as well as through gastrointestinal mucosa. The pharmaceutical compositions of embodiments of the present application show superior permeability of poorly soluble lipophilic drugs through the model human buccal tissues in comparison with the unformulated lipophilic drugs and commercial formulation of the same drugs (see the Examples section below). The experimental observation that the permeability was enhanced with the matrix of ingredients initially aimed at the improvement of the dissolution rate is a surprising finding.

### Examples

In the examples below, where the term “ratio” is used, it refers to the weight/weight ratio, except the cases where use of other units is specifically referred in the text.

### Materials

Sumatriptan base (from Manus Aktteva, India); Imitrex®, a nasal spray containing sumatriptan 20 mg as hemisulphate (from GlaxoSmithKline Inc); Emend® (aprepitant 125 mg, Merck); Sumatriptan base standard from United States Pharmacopeia (USP, Sigma-Aldrich®); Cannabidiol (from AMRI Ltd., Great Britain); Tetrahydrosumatriptan (from THC Pharm); Poloxamer 407 and Kollidone CL (from BASF, Germany); carboxymethylcellulose sodium NaCMC (Aqualon® CMC-7L2P, from Aqualon, Ashland Inc.); Modified starch (from Ingredion, USA); Stevinol (Rebaten 97, from Seppic Inc.); Mannitol and Mg Stearate (from Merck); Menthol (from Anhyi Yinfeng Pharmaceutical); strawberry and banana flavors (from Quest International India Ltd); sodium chloride and PBS (from Biological Industries, Israel); aprepitant, prednisolone, insulin, potassium phosphate monobasic and dibasic and PVP (from Sigma, Israel); lactose (from Alfa Chem, USA); silicon dioxide (from Evonik Industries, Germany); n-propanol and acetone (from Bio Lab, Israel).

### Methods

The liquid intermediates, containing the active compound(s) and the polymers, were prepared using different size glassware, magnetic plates, peristaltic pump and tubing. The spray-drying process was conducted using Mini Spray Dryer B-290 of Buchi Labortechnik AG. Tablet compression was performed with a Mini 8-D tablet press Dynamic Exim Co. Ltd. Granules preparation was performed with Dynamic Exim dry granulator. The dissolution test was performed in accordance with USP Dissolution Method <711> and FDA Dissolution Methods for Drug Products using the paddle apparatus Pharma Test model DT70 equipped with 1L and 250 ml vessels. The quantification was performed using HPLC, Dionex. Appropriate amount of prepared granules or tablets as well as control powders or tablets were dissolved in 250 ml of artificial saliva, at 37° C., with rotation speed of 75 rpm. Simulated saliva solution was prepared according to SS5 solution recipe reported in Marques et al, 2011, Dissolution Technologies, 18, 15-28. The tablets disintegration test was performed in accordance with USP Disintegration Method <701>.

Thermal properties of the compositions were studied using standard DSC equipment such as Differential Scanning calorimeter from Mettler Toledo, model DSC 820, Aluminum Crucibles standard 40 μl ME-27331, Mettler Toledo Balance MT-15, Sealer Press, Crucible handling set ME-119091, and Mettler-Toledo STAR^{e} Software System. The samples (5-10 mg) were heated at a heating rate of 10° C./min from 25° C. to 100° C.

X-ray diffraction measurements were performed using an Ultima III theta-theta diffractometer with a variable temperature control (Rigaku, Japan). Generator settings were: 40 kV, 40 mA. The detector was either a solid state module D/tex-25 or a scintillation counter. Data analysis was performed using Jade 8 or 9 analyses programs (MDI, CA). All calculations were performed with the PowderCell for Windows version 2.4 program developed by W. Kraus & G. Nolze, Federal Institute for Materials Research and Testing, Berlin, Germany. Structure solution and refinement were done by direct methods using SHELX.

Particle size of the nanodispersions was measured using Dynamic Light Scattering (DLS). The method was run on the Malvern Zen 3600, Zetasizer-nano series. The samples were prepared by suspending spray-dried powder in water (0.075-0.1%) at 25-30° C. First, water was added to the appropriate amount of the powder and the mixture was left for 15 min. Then, the suspension was magnetically stirred during 4 min at 300 rpm and 1 ml of the suspension was transferred to a cuvette for measurement. A series of at least 5 repeating measurements was carried out at 25-30° C. Concentrations of active compounds in formulations were determined by validated HPLC-UV method using Summit DI 6009 and Ultimate 3000 Dionex (Germany) HPLC systems with photodiode array (PDA) detector and Chromeleon Version 6.70 software package.

Permeability studies were performed as follows. The barrier membranes, EpiOral™ representing a highly differentiated, three-dimensional, cultured human buccal tissue equivalent were obtained from MatTek Corp (Ashland, Mass.). Samples were mounted in vertical Franz diffusion cells (PermeGear Inc., Bethlehem, Pa.). These exhibited a diffusion-available surface area of 0.64 cm² and a receptor compartment volume of 5.1 mL The receptor compartments were filled with isotonic phosphate buffered saline (0.155 M and pH 7.4), which was stirred at 600 rpm. The fluid in each receptor compartment was maintained at 37±0.5° C. by using a thermostatic water pump (Freed Electric, Haifa, Israel) that circulated water through the jacket surrounding each main chamber. The biological membranes were initially left in the Franz cells for 1 h in order to facilitate their hydration before the experiment. After this period, a 500-μL aliquot of 7.5 mg/ml sumatriptan solution/dispersion in artificial saliva was deposited in each donor compartment. The donor compartment was covered with Parafilm® to prevent evaporation. Receptor solution samples of 300 μL were then collected at 3, 6, 9, 12, 20, 40 and 60 minutes and replaced with 300 μL phosphate buffer, placed on ice and stored at −20° C. until subjected to HPLC or LCMS analysis. Each permeation experiment was conducted as four replicate runs.

### Example 1: Formulation of Sumatriptan with Poloxamer 407 and NaCMC

### Drug Solution:

Sumatriptan base (1.0 g) was dissolved in a mixture of 17 g n-propanol and 8 g acetone at 25° C. under stirring at 300 rpm.

### Polymers Solution:

Poloxamer 407 (2.0 g), NaCMC (1.0 g) and mannitol (0.5 g) were dissolved in 50 ml of water under stirring at 300 rpm at 57° C.

The drug solution was added to the polymers solution at a feeding rate of 2 ml/min, under stirring at 300 rpm at 55° C. The resultant clear homogeneous hot (50-55° C.) solution was spray dried, using Buchi Mini Spray Drier with inlet air temperature 105° C. and outlet temperature 62° C., thus obtaining a powder.

### Example 2: Formulation of Sumatriptan with Poloxamer 407, NaCMC and Modified Starch

### Drug Solution:

Sumatriptan (1.0 g) was dissolved under stirring at 300 rpm in a mixture of 17 g n-propanol and 8 g acetone at 25° C.

### Polymers Solution:

Poloxamer 407 (2.0 g), NaCMC (0.5 g), modified starch (0.5 g) and mannitol (0.5 g) were dissolved in 50 ml of water under stirring at 300 rpm at 57° C.

The drug solution was added to the polymers solution at a feeding rate of 2 ml/min, under stirring at 300 rpm and at 55° C. The resultant clear homogeneous hot (50-55° C.) solution was spray dried, using Buchi Mini Spray Drier with inlet air temperature 105° C. and outlet temperature 62° C., thus obtaining a powder.

### Example 3: Formulation of Sumatriptan with Poloxamer 407, NaCMC, Modified Starch and Potassium Phosphate Monobasic

### Drug Solution:

Sumatriptan (1.0 g) was dissolved under stirring at 300 rpm in a mixture of 17 g n-propanol and 8 g acetone at 25° C.

### Polymers Solution:

Poloxamer 407 (2.0 g), NaCMC (0.5 g), modified starch (0.5 g), mannitol (0.5 g), stevinol (1.0 g) and KH₂PO₄ (1.5 g) were dissolved in 50 ml of water under stirring at 300 rpm at 57° C.

The drug solution was added to the polymers solution at a feeding rate of 2 ml/min, under stirring at 300 rpm and at 55° C. The resultant clear homogeneous hot (50-55° C.) solution was spray dried using Buchi Mini Spray Drier with inlet air temperature 105° C. and outlet temperature 56° C., thus obtaining a powder.

### Example 4: Solvent-Free Preparation of the Formulation of Sumatriptan with Poloxamer 407, NaCMC, Modified Starch and Potassium Phosphate Monobasic

Poloxamer 407 (2.0 g), NaCMC (0.5 g), modified starch (0.5 g) mannitol (0.5 g), stevinol (1.0 g) and KH₂PO₄ (1.5 g) were dissolved in 50 ml of water under stirring at 300 rpm at 57° C. Sumatriptan base (1 g) was added to the polymers solution under stirring at 300 rpm and at 55° C. The resultant clear yellowish homogeneous hot (50-55° C.) solution was spray dried, using Buchi Mini Spray Drier with inlet air temperature 115° C. and outlet temperature 54° C., thus obtaining a powder.

### Example 5: pH Measurements of Sumatriptan Formulations

100 mg of the formulation of Example 1 or Example 4 were dissolved in 5 ml of deionized (DI) water and pH was measured for obtained solution. The pH of the solution of the formulation obtained in Example 1 was 10.1 and pH of the solution of the formulation obtained in Example 4 was 7.1. The results justify the need of pH adjustment and addition of a buffering agent (KH₂PO₄) to the formulation as carried out in Example 3.

### Example 6: Dissolution Rate of Compositions from Examples 1-3

Unformulated sumatriptan base and powders of the formulations of Examples 1-3 were subjected to a dissolution test in simulated saliva. The drug loading in the first set of experiments was 2.5 mg/ml. Results are summarized in Table 1.

TABLE 1
Sumatriptan dissolution at drug loading 2.5 mg/ml

| | Unformulated | Example 1 | Example 2 |
|---|---|---|---|
| | sumatriptan | Drug | Drug |
| Time | Drug released | released | released |
| (min) | (%) | (%) | (%) |
| 10 | 25 | 50 | 66 |
| 20 | 25 | 50 | 75 |
| 40 | 33 | 58 | 84 |
| 60 | 35 | 62 | 92 |

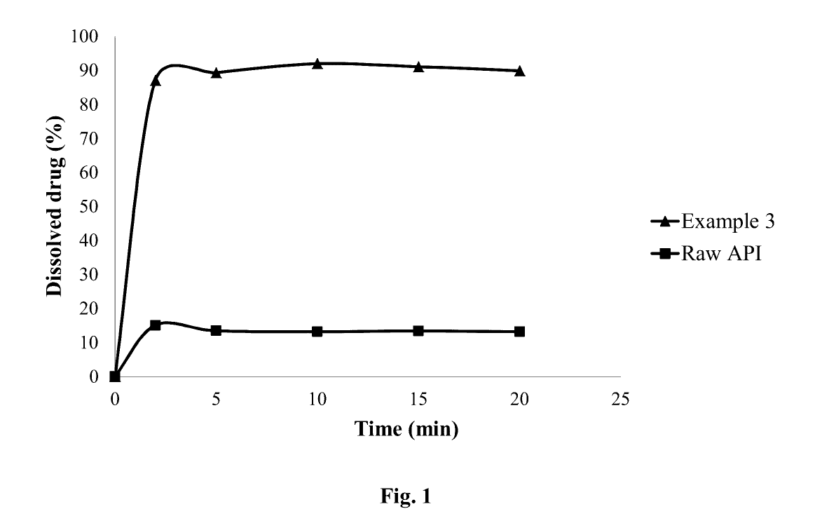

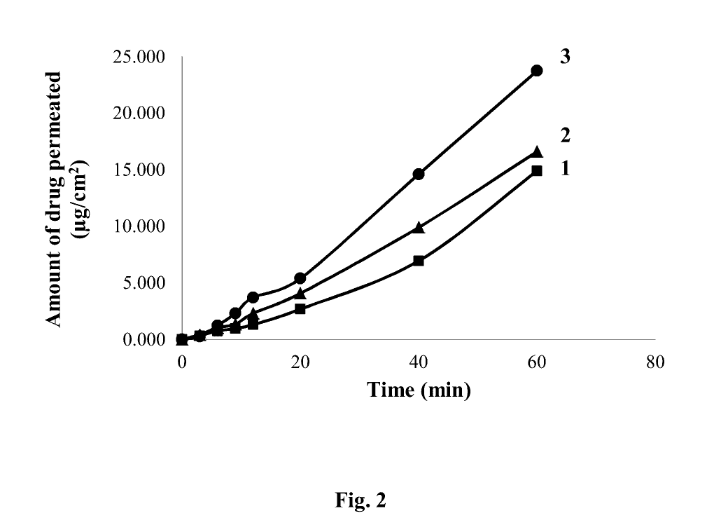

In order to simulate the real dose administration, drug dissolution with API loading of 10 mg/ml was tested. The results are summarised in Table 2 and FIG. 1, which shows the dissolution profile of Sumatriptan API from the Transmucosal formulation of an embodiment (trinagles) vs unformulated sumatriptan in saliva (squares).

TABLE 2
Sumatriptan dissolution at drug loading of 10 mg/ml

| Time | Unformulated API | Example 3 |
|---|---|---|
| (min) | Drug released (%) | Drug released (%) |
| 5 | 13 | 66 |
| 10 | 13 | 81 |
| 20 | 13 | 95 |

Table 2 and FIG. 1 clearly demonstrate the superior solubility of the composition of Example 3 over unformulated sumatriptan.

### Example 7: Thermal and Structural Properties of Sumatriptan Formulations

In order to determine the thermal properties of sumatriptan in the compositions of the invention, the temperature and the enthalpy of melting of spray-dried powders were determined by Differential Scanning calorimetry (DSC) as described in the Methods section. These characteristics were compared to thermograms of starting commercial unformulated sumatriptan base. The enthalpy of sumatriptan melting was normalized to drug assay in each composition and given in Joule per gram of sumatriptan. The results are summarised in Table 3.

TABLE 3
DSC of sumatriptan formulations

| | Tₘ | ΔHₘ |
|---|---|---|
| Sample | (° C.) | (J/g_{FF}) |
| Unformulated | 176 | 450 |
| sumatriptan base | | |
| Example 1 | 158 | 55 |
| Example 2 | 157 | 28 |
| Example 3 | 152 | 6 |
| Example 4 | 146 | 5 |

As can be seen from Table 3, unformulated crystalline sumatriptan powder exhibited an endothermic peak around 176° C. with melting enthalpy of 450 J/g. In contrast, introduction of sumatriptan into the polymers-sumatriptan complex according to the invention and its interactions with polymers and other ingredients resulted in a significant depression of the drug fusion peak. The compositions described in Examples 1 and 2 demonstrate 8-16 fold reduction correspondingly and drug in formulations of Examples 3 and 4 demonstrate 75-91 folds of enthalpy compared to the bulk starting sumatriptan. More specifically, DSC data shows 6-fold reduction of sumatriptan enthalpy for the solid dispersion described in Example 1. The maximum degree of interaction is exhibited by sumatriptan with Poloxamer 407, NaCMC, starch and KH₂PO₄ in the ratio 2:4:1:1:1.5 (Examples 3 and 4), where only residual peak of drug is observed. The thermotropic profile of the compositions of the invention also pointed out strong interactions of the sumatriptan with the polymers and weak acid. The temperature of melting is shifted down from 176° C. to 146° C. in Examples 1-4.

The XRD analysis demonstrates that most characteristics peaks of raw crystalline sumatriptan with 20 of 7.3°, 14.6°, 17.4°, 18.7° and 19.0° are preserved in the formulations of invention.

### Example 8: Permeability of Sumatriptan Through Human Buccal Membrane

The permeability of unformulated sumatriptan base, of API (in the base form) released from the composition of the present invention (Example 3) and of the commercial formulation Imitrex® was tested using EpiOral™ buccal tissues as described in the Methods section. EpiOral™ buccal tissues consist of normal, human-derived epithelial cells that have been cultured to form multilayered, highly differentiated models of the human buccal phenotypes. The sumatriptan (STP) permeability results are summarized in Table 4 and FIG. 2, which shows shows the Franz diffusion cell experiment for testing in-vitro permeability through human buccal tissues of three sumatriptan samples suspended in 0.5 ml of artificial saliva at concentration 7.5 mg/ml:

(1) Unformulated sumatriptan (squares),

(2) Imitrex® nasal spray containing Sumatriptan API (triangles), and

(3) Sublingual formulation of an embodiment (see Example 3) (circles).

TABLE 4
Permeability tests

| | STP product | | |
|---|---|---|---|
| | STP Permeability in μg/cm² | | |
| Time point | Unformulated | Imitrex ® | |
| (min) | STP | nasal spray | Example 3 |
| 0 | 0.000 | 0.000 | 0.000 |
| 3 | 0.330 | 0.414 | 0.269 |
| 6 | 0.736 | 1.027 | 1.227 |
| 9 | 0.964 | 1.339 | 2.295 |
| 12 | 1.295 | 2.299 | 3.708 |
| 20 | 2.671 | 4.068 | 5.378 |
| 40 | 6.938 | 9.892 | 14.602 |
| 60 | 14.887 | 16.593 | 23.738 |

The data above clearly demonstrate that sumatriptan released from the composition of the present invention possesses a better flux rate through buccal membrane than raw STP or the commercial STP formulation (Imitrex®). In fact, the sumatriptan diffusion coefficient was increased in the composition of the invention without use of a penetration enhancer. This observation is surprising and totally unexpected.

### Example 9: Preparation of Formulation with High Loading of Sumatriptan Base

Poloxamer 407 (2.8 g), NaCMC (1.0 g), modified starch (2.9 g) Avicel PH 101 (0.6 g), rebaten (3.0 g)) were dissolved in 100 ml of water under stirring at 300 rpm at 57° C. Sumatriptan base (11.5 g) and KH₂PO₄ (7.0 g) were dissolved in 100 ml of water and added to the polymers solution under stirring at 300 rpm and at 55° C. The resultant clear yellowish homogeneous hot (50-55° C.) solution was spray dried, using Buchi Mini Spray Drier with inlet air temperature 145° C. and outlet temperature 65° C., thus obtaining a powder.

### Example 10: Preparation of Sublingual Tablets with Active Doses of 25, 50 and 75 mg Sumatriptan

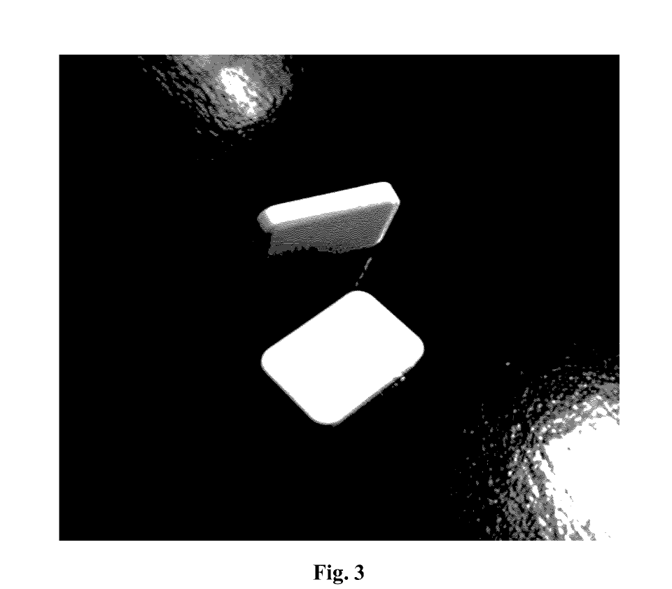

The composition of the invention described in Example 4 was first mixed with the excipients listed in Part 1 of Table 5, then the excipients from Part 2 were added to the mixture, mixed together and compressed to tablets shown in FIG. 3. These tablets have flat, rectangle form in order to fit the under tong cavity and possess the relatively short disintegration and dissolution time under the tongue (5-7 min) needed for transmucosal delivery. Taste-masking agents i.e. sweeteners (sucralose, rebaten), flavours (vanillin) and menthol are added into tablet composition in order to musk the bitter taste of sumatriptan.

TABLE 5
Components of tablets

| | | Weight per | Weight per | Weight per |
|---|---|---|---|---|
| | | tablet of | tablet of | tablet of |
| | | sumatriptan | sumatriptan | sumatriptan |
| | Ingredients | 25 (mg) | 50 (mg) | 75 (mg) |
| Part | Sumatriptan | 75.0 | 160.0 | — |
| 1 | formulation | | | |
| | from Example 4 | | | |
| | Sumatriptan | — | — | 210 |
| | formulation | | | |
| | from Example 9 | | | |
| | Mannitol DC | 50.0 | 10.0 | 13.0 |
| | Rebaten | 39.0 | 70.0 | 70.0 |
| | Menthol | 1.0 | 2.0 | 2.0 |
| | Vanillin | 2.0 | — | — |
| | Kollidon CL | 10.0 | 14.0 | 15.0 |
| | Aerosil 200 | 1.0 | 5.0 | 5.0 |
| | Avicel pH 101 | 10.0 | 14.0 | 15.0 |
| | Sucralose | 10.0 | 20.0 | 15.0 |
| Part | Mg stearate | 2.7 | 4.0 | 4.0 |
| 2 | Yellow color | 0.3 | 1.0 | 1.0 |

### Example 11: Dissolution of Sublingual Sumatriptan Tablets

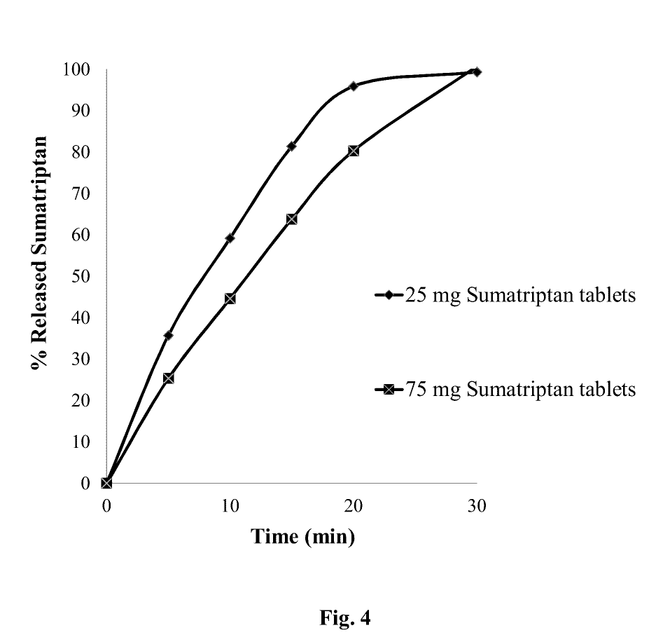

Six tablets of each dose were subjected to dissolution test in buffer pH 6.8 (simulated saliva solution), rotational speed 100 rpm, temperature 37° C. The results are summarised in Table 6 and FIG. 4, which shows the dissolution profile of sumatriptan base flat-square sublingual tablets with active dose of 25 mg (rhombs) and 75 mg (squares) in artificial saliva.

TABLE 6
Sumatriptan sublingual tablets dissolution

| Time point (min) | | | | | | | AVG |
|---|---|---|---|---|---|---|---|
| | 25 mg sumatriptan tablets | | | | | | |
| 5 | 35.1 | 34.3 | 33.7 | 37.4 | 36.0 | 37.9 | 35.7 |
| 10 | 61.7 | 58.6 | 59.1 | 65.4 | 54.6 | 55.6 | 59.2 |
| 15 | 76.1 | 79.4 | 79.6 | 86.2 | 84.2 | 82.7 | 81.4 |
| 20 | 97.0 | 94.3 | 93.7 | 99.1 | 93.1 | 98.6 | 95.9 |
| 30 | 99.8 | 98.7 | 100.4 | 98.7 | 100.0 | 98.0 | 99.3 |
| | 75 mg sumatriptan tablets | | | | | | |
| 5 | 22.5 | 25.2 | 26.5 | 26.0 | 25.5 | 26.2 | 25.3 |
| 10 | 39.8 | 46.1 | 43.7 | 45.9 | 46.4 | 45.5 | 44.6 |
| 15 | 57.2 | 64.6 | 65.8 | 67.1 | 64.8 | 63.5 | 63.8 |
| 20 | 72.6 | 80.9 | 82.9 | 81.2 | 82.5 | 81.7 | 80.3 |
| 30 | 95.2 | 103.3 | 93.0 | 102.8 | 108.1 | 101.6 | 100.7 |

### Example 12: Preparation of Sumatriptan Sublingual Granules

The composition described in Example 4 was compressed to granules with size of 1 mm using mini-tablets punches and dies. The resulting granules were dissolved in simulated saliva solution within 1-2 minutes.

### Example 13: Formulation of Cannabidiol with Poloxamer 407, NaCMC and Starch

### Drug Solution:

Cannabidiol (1.0 g) was dissolved under stirring at 300 rpm in 25 g n-propanol at 25° C.

### Polymers Solution:

Poloxamer 407 (2.0 g), NaCMC (0.5 g), starch (0.5 g) and mannitol (0.5 g) were dissolved in 50 ml water under stirring at 300 rpm at 57° C.

The drug solution was added to the polymers solution at a feeding rate of 2 ml/min, under stirring at 300 rpm and at 55° C. The resultant clear homogeneous hot (50-55° C.) solution was spray dried, using Buchi Mini Spray Drier with inlet air temperature 105° C. and outlet temperature 62° C., thus obtaining a powder.

### Example 14: Particle Size of Aqueous Dispersions Obtained from Cannabidiol Formulation

The powders produced as described in Example 13 have been suspended in deionized water as described in the Methods section. The powders of Example 13 comprising cannabidiol-polymers formulations were converted to a colloidal dispersion with particles size in the nanoscale range. The results are summarised in Table 7.

TABLE 7
Particle size measurements for cannabidiol
formulations of Example 13

| Record | Type | Sample name | Z-Ave (nm) |
|---|---|---|---|
| 1 | Size | Example 13-1 | 175.6 |
| 2 | Size | Example 13-2 | 173.5 |
| 3 | Size | Example 13-3 | 173.6 |
| 4 | Size | Example 13-4 | 172.4 |
| 5 | Size | Example 13-5 | 174 |
| Mean 1-5 | | | 173.8 |
| Std Dev | | | 1.158 |

### Example 15: Dissolution Rate of Cannabidiol Compositions

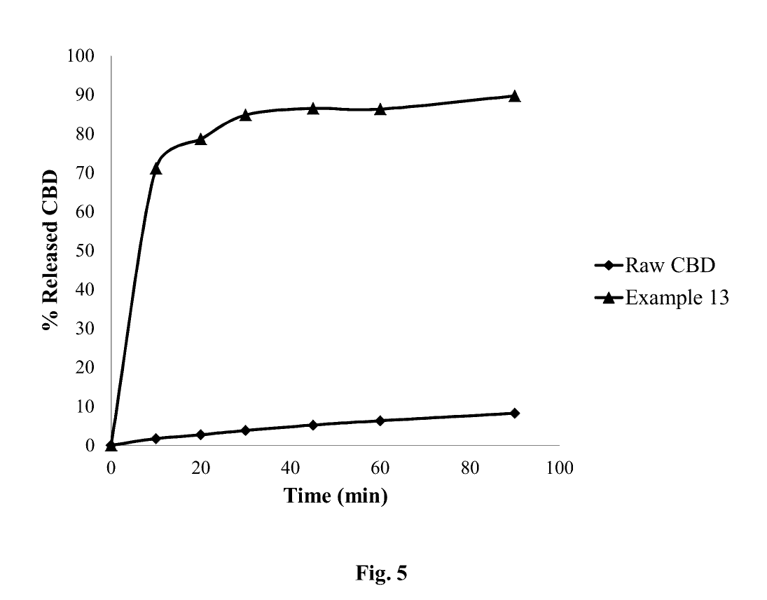

Unformulated cannabidiol (CBD) and composition of the present invention (Example 13) were subjected to a dissolution test in 200 ml of Fasted State Simulating Intestinal Fluid (FaSSIF). The drug loading was 20 mg. The obtained results are summarised in Table 7 and FIG. 5, which shows the dissolution profile of cannabidiol API from the SoluCBD™ formulation of an embodiment (see Example 13) (trinagles) vs unformulated cannabidiol in saliva (rhombs).

TABLE 8
Dissolution profile of cannabidiol composition

| Time | Unformulated CBD | Example 13 |
|---|---|---|
| 0 | 0 | 0 |
| 10 | 1.7 | 71.1 |
| 20 | 2.7 | 78.6 |
| 30 | 3.8 | 84.8 |
| 45 | 5.2 | 86.5 |
| 60 | 6.3 | 86.3 |
| 90 | 8.2 | 89.7 |

As can be seen from Examples 14 and 15, high surface area and interaction with polymers speed release and increase saturation solubility of the active compound. The dissolution rate in the FaSSIF of the composition (Example 14) is 11-folds higher than the dissolution rate of the unformulated CBD compound.

### Example 16: Formulation of Aprepitant with Poloxamer 407, NaCMC and PVP

### Drug Solution:

Aprepitant (1.4 g) was dissolved under stirring at 300 rpm in 80 g of n-propanol at 50° C.

### Polymers Solution:

Poloxamer 407 (2.0 g), NaCMC (1.4 g) and PVP (0.14 g) were dissolved in 50 ml of water under stirring at 300 rpm at 50° C.

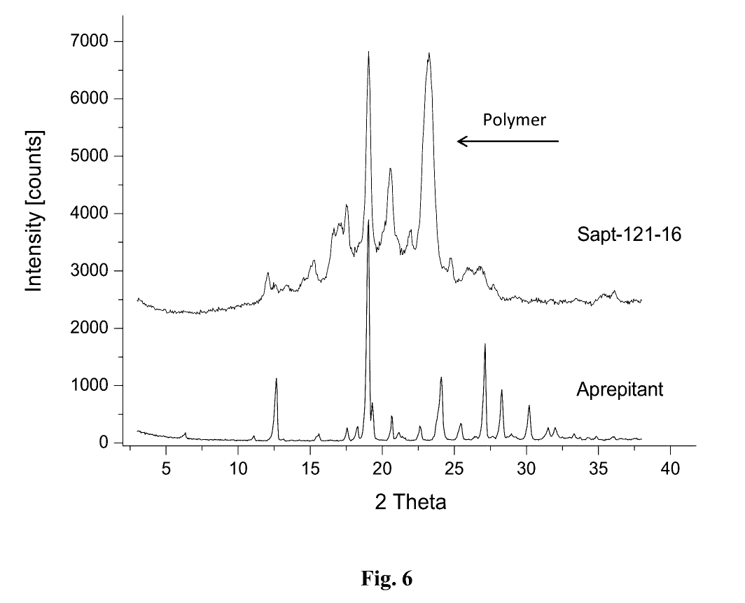

The drug solution was added to the polymers solution at a feeding rate of 2 ml/min, under stirring at 300 rpm and at temperature 55° C. The resultant clear homogeneous hot (50-55° C.) solution was spray dried, using Buchi Mini Spray Drier with inlet air temperature 108° C. and outlet temperature 58° C., thus obtaining a crystalline powder. FIG. 6 shows the XRD (X-Ray Diffraction) spectra of Aprepitant API from the formulation of an embodiment (upper spectrum) and unformulated aprepitant (lower spectrum), proving the crystalline nature of aprepitant in the present formulation.

### Example 17: Thermal Properties of Aprepitant Formulations

The temperature and the enthalpy of melting of aprepitant as unformulated compound and as spray-dried powders were determined by Differential Scanning calorimetry (DSC). The melting temperature of aprepitant in the composition of invention is 230.3° C. and the enthalpy is 54.9 J/g. These values are essentially lower than those of unformulated aprepitant (254.3° C. and 109.2 J/g).

### Example 18: Aprepitant Dissolution Rate

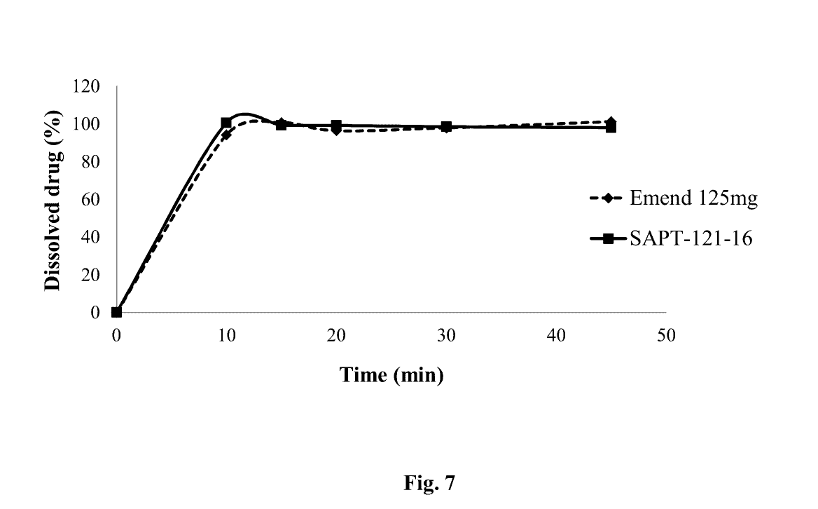

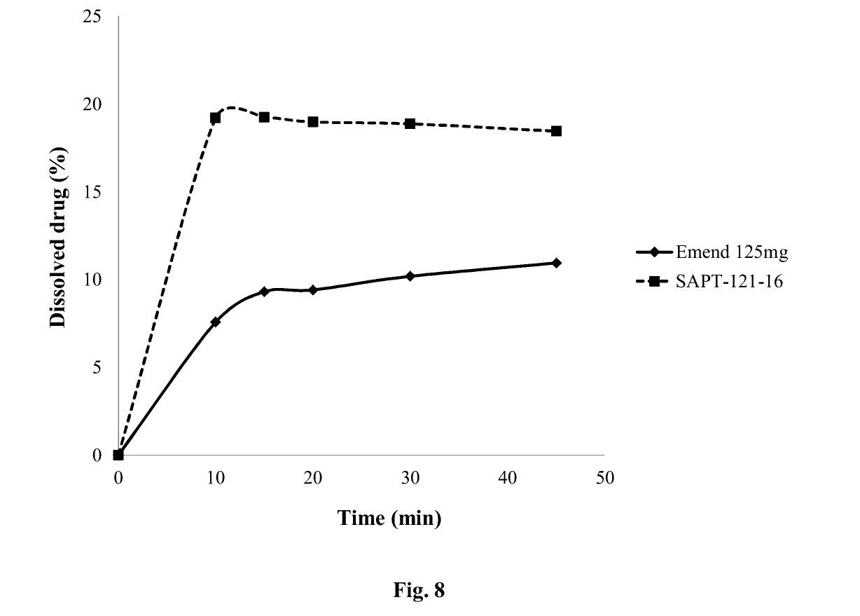

The dissolution rate of aprepitant released from the powdered formulation of an embodiment of the present application was compared to those of Emend® commercial formulations, in which aprepitant is presented in a nanocrystalline form. FIG. 7 shows the dissolution profile of aprepitant API from the formulation of an embodiment (see Example 16) (squares) vs aprepitant API from the Emend® commercial grannular formulation, in which aprepitant is presented in a nanocrystalline form (rhombs). The dissolution conditions used were similar to the conditions proposed by FDA. FIG. 8 shows the dissolution profile of aprepitant API from the formulation of an embodiment in Fasted State Simulating State Intestinal Fluid (FaSSIF) (see Example 16) (squares) vs aprepitant API from the Emend® commercial grannular formulation, in which aprepitant is presented in a nanocrystalline form (rhombs). The release rate of aprepitant from the composition of an embodiment was similar to the commercial nano-formulation in 2.2% sodium lauryl sulfate (see FIG. 7). However, the release rate of aprepitant from the formulation of an embodiment was found to be higher in FaSSIF (see FIG. 8), confirming the enhancement of the drug solubility in comparison with the commercial formulation.

### Example 19: Oral Mucosal Formulation of Tetrahydrocannabinol

### Drug Solution:

Tetrahydrocannabinol (THC) (1.0 g) and D-tocopherol polyethylene glycol (1 g) antioxidant was dissolved under stirring at 300 rpm in 8 g ethanol at 30° C.

### Polymers Solution:

Mixture of Poloxamer 407 (2.0 g), NaCMC (1.0 g), soluble starch (1.0 g) and stevinol (1.0 g) were dissolved in 40 ml of water under stirring at 300 rpm at 57° C. 0.5 g of microcrystalline cellulose was added to the obtained clean solution.

The drug solution was added to the polymer solution at a feeding rate of 2 ml/min, under stirring at 300 rpm and at temperature 45° C. The resultant clear homogeneous solution was spray dried from hot (50-55° C.) solution, using Buchi Mini Spray Drier with inlet air temperature 105° C. and outlet temperature 62° C., thus obtaining a powder.

### Example 20: Particle Size of Aqueous Dispersions Obtained from THC Formulation

The powders produced as described in Example 19 have been suspended in deionised water as described in the Methods section. The powders of Example 19 comprising the THC-polymers formulations were converted to colloidal dispersions with the particles size in the nanoscale range. The results are summarised in Table 9.

TABLE 9
Results of particle size measurements for THC formulation

| Record | Type | Sample name | Z-Ave (nm) |
|---|---|---|---|
| 1 | Size | Example 19-1 | 63.84 |
| 2 | Size | Example 19-2 | 62.42 |
| 3 | Size | Example 19-3 | 122.60 |
| 4 | Size | Example 19-4 | 95.85 |
| 5 | Size | Example 19-5 | 61.08 |
| Mean 1-5 | | | 81.16 |
| Std Dev | | | 27.33 |

### Example 21: Bioavaliability Pharmacokinetic Study to Compare Sumatriptan Transmucosal Sublingual Tablet with Commercial Imitrex® Sumatriptan Oral Tablet in Healthy Volunteers

A bioavailability test of the sumatriptan sublingual tablet 75 mg of Example 10 and commercial Imitrex® sumatriptan oral tablet 100 mg was carried out in human patients as follows. A randomised two-way crossover comparative bioavailability study was conducted with a single administration of each drug in 3 healthy volunteers in the fasted state. One week following the first administration, the volunteers received the alternative treatment according to a predetermined randomisation table. A 7-day washout between periods was maintained before dosing the next product. Blood samples were collected in each period at 0, 5, 15, 30, 45, 60, 90 and 120 minutes in order to characterise the drug pharmacokinetic profile: Tₘₐₓ, Cₘₐₓ and AUCₜ (area under the concentration-time curve from zero up to a definite time t, the parameter that is used as an index of the drug exposure of the body, when referred to the plasma drug levels, and is closely dependent on the drug amount that enter into the systemic circulation). These samples were analysed for sumatriptan content by a LC-MS/MS method. The volunteers reported that sumatriptan sublingual tablet 75 mg was disintegrated under the tongue within 5-7 minutes. The obtained pharmacokinetic parameters of the test and the reference products are shown below in Table 10.

TABLE 10
Sumatriptan pharmacokinetic parameters in human patients

| | | | | Cₘₐₓₜₑₛₜ/ | AUCₜₑₛₜ/ |
|---|---|---|---|---|---|
| | AUC_{0-2 h} | Tₘₐₓ | Cₘₐₓ | C_{maxref} | AUC_{ref} |
| Sumatriptan Product | (ng · h/ml) | (h) | (ng/ml) | (%) | (%) |
| Sublingual Tablet 75 mg | 3.114 | 1.5 | 42 | 103 | 124 |
| Oral Tablet 100 mg | 2.505 | 1.5 | 41 | | |

The mean pharmacokinetic profile of the three volunteers and the pharmaco-kinetic profile of each volunteer following the administration of test and the reference products are presented in FIGS. 9 and 10, respectively. Specifically, FIG. 9 shows the pharmacokinetic profile of sumatriptan (the means of plasma sumatriptan values of three volunteers) following administration of sumatriptan sublingual tablet vs. Imitrex® in a crossover clinical trial. FIG. 10 shows the pharmacokinetic profile of sumatriptan (the separated profiles for each volunteer) following administration of sumatriptan sublingual tablet vs Imitrex® in a crossover clinical trial.

As can be seen from Table 10 and FIG. 10, the Tₘₐₓ and the Cₘₐₓ of the sumatriptan sublingual tablet and of Imitrex® products are similar. The AUC₀₋₂ₕ of the test drug is 124% higher than that of the reference, indicating a similar or better bioavailability of sumatriptan sublingual tablet compared to Imitrex®. Intriguingly, the mean sumatriptan plasma values and the separated values per each volunteer, as seen in FIGS. 9 and 10, respectively, exhibit an immediate increase in sumatriptan concentration following the sublingual tablet administration within the first 30 min from the drug administration, which is absent following Imitrex® administration. The immediate increase in plasma sumatriptan concentrations suggests fast transmucosal absorption, as it is observed following the transmucosal tablet administration solely. The initial increase is later followed by the Cₘₐₓ, exhibiting the gastrointestinal absorption, which is seen both in the transmucosal and in the oral (Imitrex®) formulations.

The immediate increase in plasma concentrations following the transmucosal tablet is similar to that of the intranasal formulation (Obaidi et al, Headache 2013), yet with a higher sumatriptan concentration compared to the intranasal administration, i.e. between 15-30 min sumatriptan concentrations reach up to 18 ng/ml on average following the transmucosal formulation, while the intranasal formulation reaches up to 10 ng/ml at this time points. As the intranasal formulation has been reported to have a more rapid onset of effect (Fuseau et al. Drug Disposition 2002), attributed to the rapid increase in blood levels, the present results suggest that sumatriptan sublingual tablet also has a fast headache relief onset of action.

### Example 22: Anti-Migraine Effect of Sumatriptan Transmucosal Sublingual Tablet

A 49-year-female patient suffered from chronic migraine, long term user of Imitrex® intranasal 20-mg spray and 100-mg oral tablets. The patient complained on the delayed onset of action of Imitrex® tablets and high cost and insufficient robustness of anti-pain effect of intranasal Imitrex®, since after two hours of administration secondary attack frequently appeared. The patient was given sumatriptan 75 mg sublingual tablet of an embodiment of the present application at relatively late stage of migraine attack after aura and pain spasm. Reported results: Patient reported that the tablet was dissolved under the tongue at seven minutes and pain was relieved within 15 minutes. No secondary attack was appeared.

### Example 23: Crossover Bioavaliability Study Design to Compare THC/CBD Sublingual Tablet Versus Commercial THC/CBD Oromucosal Spray Sativex® in Healthy Volunteers

Twenty healthy volunteers will randomly receive the equivalent doses of either the commercial oromucosal spray (Sativex®) or the CBD/THC oromucosal sublingual tablet of an embodiment in fasting conditions. Ten days following the first administration, the volunteers will receive the alternative treatment. Blood samples will be collected at each visit at pre-dose and at 0.25, 0.5, 0.75, 1, 1.5, 2, 3, 4, 6, 8, 12, 24, 36 and 48 hours post drug administration. Volunteers will remain at the clinical unit after dosing under observation through the collection of all the samples.

### Example 24: Formulation of Prednisolone with Poloxamer 407, NaCMC and Mannitol

### Drug Solution:

Prednisolone (1.0 g) was dissolved in the mixture of 20.4 g ethanol and 16.4 g acetone under stirring at 300 rpm.

### Polymers Solution:

NaCMC (1.0 g), Mannitol (0.5 g) and Poloxamer 407 (2.0 g) were dissolved under stirring at 300 rpm in water (50 g) at 45° C.

The drug solution was added to the polymers solution at a feeding rate of 10 ml/min, under stirring at 300 rpm at 45° C. The resulting transparent solution was spray dried using Buchi Mini Spray Drier with inlet air temperature 92° C. and outlet temperature 64° C., thus yielding a free-flowing powder. The solubility of formulated powder was increased 1.5 folds in comparison with raw material.

### Example 25: Formulation of Insulin with EDTA, Poloxamer 407 and NaCMC

NaCMC (600 mg), Poloxamer 407 (10 mg) and EDTA (1200 mg) were dissolved under stirring at 300 rpm in water (30 g) at 45° C. Insulin powder (96 mg) was dispersed in the solution, heated up to 38° C. and mixed with 9.8 g of ethanol for 10 min. The resulting opalescent solution was spray dried using Buchi Mini Spray Drier with inlet air temperature 81° C. and outlet temperature 57° C., thus yielding a free-flowing powder. The produced powder was suspended in deionized water and measured for particle size distribution. The resulted particle size mean is 566 nm.

### Example 26: Study Design for Permeability of Insulin Through Human Buccal Membrane

The permeability of raw Zn insulin and of API (in the base form) released from the nano-particulate composition of the present invention (Example 24) will be tested using EpiOral™ buccal tissues as described in the Methods section. EpiOral™ buccal tissues consist of normal, human-derived epithelial cells that have been cultured to form multilayered, highly differentiated models of the human buccal phenotypes. The assay of penetrated insulin will be tested at 0, 3, 9, 12, 15, 20, 40 and 60 min.

While certain features of the present application have been illustrated and described herein, many modifications, substitutions, changes, and equivalents will be apparent to those of ordinary skill in the art. It is, therefore, to be understood that the appended claims are intended to cover all such modifications and changes as fall within the true spirit of the present application.

1. A pharmaceutical composition for transmucosal and oral mucosal administration of an active lipophilic compound, said composition comprising:
(a) a lipophilic active compound;
(b) a polymeric matrix formed by two or more water-soluble polymers,
wherein
(i) at least one of said two or more water-soluble polymers is an amphiphilic polymer and at least one other of said two or more water-soluble polymers is either a hydrophilic polymer or an amphiphilic polymer with a hydrophobic-hydrophilic balance different from the first amphiphilic polymer; and
(ii) said polymeric matrix is not crosslinked and no covalent interaction occurs between the two or more polymers and between the polymers and the lipophilic active compound, which is interwoven with said polymeric matrix; and
(c) a rapid dissolution agent.

2. The pharmaceutical composition according to claim 1, wherein said lipophilic active compound is selected from analgesics, anti-inflammatory agents, antihelminthics, anti-arrhythmic agents, anti-bacterial agents, anti-viral agents, anti-coagulants, anti-depressants, anti-diabetics, anti-epileptics, anti-fungal agents, anti-gout agents, anti-hypertensive agents, anti-malarials, anti-migraine agents, anti-muscarinic agents, anti-neoplastic agents, chemotherapeitic drugs, antiproliferative, erectile dysfunction improvement agents, immunosuppressants, anti-protozoal agents, anti-thyroid agents, anxiolytic agents, sedatives, hypnotics, neuroleptics, beta-blockers, cardiac inotropic agents, corticosteroids, diuretics, anti-parkinsonian agents, gastro-intestinal agents, histamine receptor antagonists, keratolyptics, lipid regulating agents, anti-anginal agents, Cox-2 inhibitors, leukotriene inhibitors, macrolides, muscle relaxants, nutritional agents, opiod analgesics, protease inhibitors, sex hormones, stimulants, muscle relaxants, anti-osteoporosis agents, anti-obesity agents, cognition enhancers, anti-urinary incontinence agents, anti-benign prostate hypertrophy agents, essential fatty acids, non-essential fatty acids, and mixtures thereof.

3. The pharmaceutical composition according to claim 1, wherein said lipophilic active compound is selected from acetretin, acyclovir, albendazole, albuterol, almotriptan, aminoglutethimide, amiodarone, amlodipine, amphetamine, amphotericin B, amprenavir, aprepitant, atorvastatin, atovaquone, azithromycin, aztreonum, baclofen, beclomethasone, benezepril, benzonatate, betamethasone, bicalutanide, budesonide, bupropion, busulfan, butenafine, calcifediol, calcipotriene, calcitriol, camptothecin, candesartan, cannabidiol, capsaicin, carbamezepine, carotenes, cefixime, cefuraxime axetil, celecoxib, cerivastatin, cetirizine, chlorpheniramine, cholecalciferol, cilostazol, cimetidine, cinnarizine, ciprofloxacin, cisapride, clarithromycin, clemastine, clomiphene, clomipramine, clopidogrel, codeine, coenzyme Q10, cyclobenzaprine, cyclosporin, danazol, dantrolene, dexchlor-pheniramine, diclofenac, dicoumarol, digoxin, dehydroepiandrosterone, dihydro-ergotamine, dihydrotachysterol, dirithromycin, donezepil, enlimomab, efavirenz, eletriptan, eprosartan, ergocalciferol, ergotamine, essential fatty acid sources, etodolac, etoposide, famotidine, cannabidiol, fentanyl, fexofenadine, finasteride, fluconazole, flurbiprofen, fluvastatin, fosphenyloin, frovatriptan, fuirazolidone, gabapentin, gemfibrozil, glibenclamide, glipizide, glyburide, glimepiride, griseofulvin, halofantrine, hydrocortizone, ibuprofen, indinavir, irbesartan, irinotecan, isosorbide dinitrate, isotretinoin, itraconazole, ivermectin, ketoconazole, ketorolac, lamotrigine, lansoprazole, leflunomide, lidocaine, lisinopril, loperamide, loratadine, lovastatin, L-thryroxine, lutein, lycopene, medroxyprogesterone, mifepristone, mefloquine, megestrol, methadone, methoxsalen, metronidazole, miconazole, midazolam, miglitol, minoxidil, mitoxantrone, montelukast, morphine, nabumetone, nalbuphine, naratriptan, nelfinavir, nifedipine, nilsolidipine, nilutanide, nitrofurantoin, nizatidine, omeprazole, oprevelkin, oestradiol, oxaprozin, oxibutonine, paclitaxel, paracalcitol, paroxetine, pantoprazole, pentazocine, pioglitazone, pizofetin, phenoxymethyl penicillin, pravastatin, prednisolone, probucol, progesterone, propofol, pseudoephedrine, pyridostigmine, rabeprazole, raloxifene, rofecoxib, repaglinide, rifabutine, rifapentine, rimexolone, ritanovir, rizatriptan, rosiglitazone, saquinavir, sertraline, sibutramine, sildenafil, simvastatin, sirolimus, spironolactone, sumatriptan, svitriptan, tacrine, tacrolimus, tamoxifen, tamsulosin, targretin, tazarotene, telmisartan, teniposide, terbinafine, terazosin, tetrahydrocannabinol, tiagabine, ticlopidine, tirofibran, tizanidine, topiramate, topotecan, toremitfene, tramadol, tretinoin, troglitazone, trovafloxacin, ubidecarenone, valsartan, venlafaxine, verteporfin, vigabatrin, vitamin A, vitamin D, vitamin E, vitamin K, zafirlukast, zileuton, zolmitriptan, zolpidem or zopiclone, and pharmaceutically acceptable salts, isomers, and mixtures thereof.

4. The pharmaceutical composition according to claim 1, wherein said lipophilic active compound is a cannabinoid selected from tetrahydrocannabinol (THC) and cannabidiol (CBD); or a triptan drug selected from almotriptan, eletriptan, frovatriptan, naratriptan, rizatriptan, sumatriptan, svitriptan, zolmitriptan, fentanyl, morphine, oxibutonine, tramadol, aprepitant, testosterone, prednisolone, sildenafil, omeprazole, lansoprazole, pantoprazole, insulin, HGF, octreotide and glucagon.

5. The pharmaceutical composition according to claim 1, wherein said rapid dissolution agent is mannitol, stevinol, polyvinylpyrrolidones (PVP), ethylenediaminetetra-acetic acid (EDTA), or a mixture thereof.

6. The pharmaceutical composition according to claim 1, further comprising one or more optional pharmaceutically acceptable carriers, excipients, additives or combinations thereof, wherein said additives are selected from buffering or pH-adjusting agents, taste-masking agents and disintegrating agents, and wherein said taste-masking agents are selected from sweeteners, essential oils and common flavors.

7. The pharmaceutical composition according to claim 6, wherein independently said buffering agent is KH₂PO₄, said disintegrating agent is cross-linked starch, crosscarmellose sodium or crosspovidone, and said taste-masking agent is a mixture of sucralose, stivenol, menthol and optionally vanillin.

8. The pharmaceutical composition according to claim 1, wherein said amphiphilic polymer is selected from polyethylene oxide (PEO), PEO derivatives, poloxamers, poloxamines, polyvinylpyrrolidones (PVP), hydroxypropyl cellulose, hypromellose, hypromellose phthalate, hypromellose acetate succinate, polyacrylates, polymethacrylates, polyethylene glycol (PEG) copolymers, PEO/polypropylene glycol copolymers, PEG-modified starches, vinyl acetate-vinyl pyrrolidone copolymers, polyacrylic acid copolymers, polymethacrylic acid copolymers, plant proteins and protein hydrolysates.

9. The pharmaceutical composition according to claim 1, wherein said hydrophilic polymer is selected from starch, soluble starch, sodium carboxymethylcellulose (NaCMC), hydroxyethylcellulose, polyvinyl alcohol, sodium alginate, chitosan, and carrageenan.

10. The pharmaceutical composition according to claim 1, wherein
(i) two polymers form the polymeric matrix, one of the polymers is an amphiphilic polymer, and the other polymer is a hydrophilic polymer;
(ii) three polymers form the polymeric matrix, two of the polymers are amphiphilic polymers, and the other polymer is a hydrophilic polymer; or
(iii) three polymers form the polymeric matrix, one of the polymers is an amphiphilic polymer, and the other two polymers are hydrophilic polymers.

11. The pharmaceutical composition according to claim 10, wherein said amphiphilic polymer is Poloxamer 407 or polyvinylpyrrolidone (PVP), and said hydrophilic polymer is sodium carboxymethyl cellulose (NaCMC) or soluble starch.

12. The pharmaceutical composition according to claim 1 having the following content:
(i) Sumatriptan, Poloxamer 407, NaCMC, and mannitol;
(ii) Sumatriptan, Poloxamer 407, NaCMC, soluble starch and mannitol;
(iii) Cannabidiol, Poloxamer 407, NaCMC and mannitol;
(iv) Aprepitant, Poloxamer 407, NaCMC and mannitol;
(v) Tetrahydrocannabinol, D-α-tocopherol, polyethylene glycol, Poloxamer 407, NaCMC, soluble starch and stevinol;
(vi) Prednisolone, Poloxamer 407, NaCMC and mannitol; or
(vii) Insulin, EDTA, Poloxamer 407 and NaCMC.

13. The pharmaceutical composition according to claim 6 having the following content:
(i) Sumatriptan, Poloxamer 407, NaCMC, mannitol and KH₂PO₄; or
(ii) Sumatriptan, Poloxamer 407, NaCMC, soluble starch, mannitol, stevinol and KH₂PO₄;

14. A method for the preparation of a composition according to claim 1, comprising the steps of:
i) preparing a clear and homogeneous solution of said two or more polymers, said rapid dissolution agent and said lipophilic active compound in water or in a mixture of water and one or more organic solvents; and
ii) drying the obtained clear and homogeneous solution, preferably by spray drying, to form a dry powder.

15. The method according to claim 14, wherein the clear and homogeneous solution in step (i) is obtained by adding the lipophilic active compound dissolved in one or more organic solvents to an aqueous solution of the polymers and the rapid dissolution agent, wherein said organic solvent is miscible with water and is not causing precipitation of said polymers when the resulted organic solution containing said lipophilic active compound is added to the polymers solution.

16. The method according to claim 15, wherein said organic solvent is acetic acid, acetonitrile, acetone, 1-butanol, 2-butanol, N,N-dimethylacetamide; N,N-dimethylformamide, dimethyl sulfoxide, 1,4-dioxane, ethanol, formic acid, methanol, 3-methyl-1-butanol, methylethyl ketone, 2-methyl-1-propanol, 1-methyl-2-pyrrolidone, 1-pentanol, n-propanol, 2-propanol, tetrahydrofuran, a mixture of n-propanol and acetone, or a mixture of ethanol and water.

17. The pharmaceutical composition of claim 1, said pharmaceutical composition being manufactured in a sublingual or buccal transmucosal solid dosage form selected from capsules, tablets, beads, grains, pills, granulates, granules, powder, pellets, sachets, troches, disks, films, oral suspensions and aerosol.

18. The pharmaceutical composition of claim 17, wherein said sublingual or buccal transmucosal solid dosage form is a tablet.

19. The pharmaceutical composition according to claim 1, wherein said lipophilic active compound is in the soluble form or in the form of a colloidal dispersion, upon contact with an aqueous medium.
